(19) United States
(12) Patent Application Publication
(10) Pub. No.: US 2024/0301490 A1
(43) Pub. Date: Sep. 12, 2024

(54) HIGH THROUGHPUT SINGLE CELL BASED ASSAY FOR CAPTURING GENOMIC INFORMATION FOR FUNCTIONAL AND IMAGING ANALYSIS AND METHODS OF USE

(71) Applicant: Zafrens, Inc., San Diego, CA (US)

(72) Inventor: Kandaswamy Vijayan, San Diego, CA (US)

(73) Assignee: Zafrens, Inc., San Diego, CA (US)

(21) Appl. No.: 18/195,049

(22) Filed: May 9, 2023

Related U.S. Application Data
(60) Provisional application No. 63/339,782, filed on May 9, 2022.

(51) Int. Cl. C12Q 1/6876; C12Q 1/6806; C12Q 1/6834; C12Q 1/6869

(52) U.S. Cl. CPC C12Q 1/6876; C12Q 1/6806; C12Q 1/6834; C12Q 1/6869

(57) ABSTRACT

A method of cellular analysis at high throughput is provided. More specifically, the invention relates to capturing single cells, culturing single cells to generate clonal copies of single cells, perturbing the single cells or the clonal copies derived from the single cells, performing experiments on single cells or clonal copies of single cells, imaging cells, capturing genomic content and other material from cells and being able to relate the captured genomic information to the functional and imaging analysis performed on the same cell.

This application claims priority to application Ser. No. 63/339,782 filed on May 9, 2022, which is incorporated herein by reference.

The patent or application file contains at least one drawing executed in color. Copies of this patent or patent application publication with color drawing(s) will be provided by the Office upon request and payment of the necessary fee.

This disclosure is related generally to cellular analysis at high throughput and more specifically, to capturing, culturing, perturbing, and performing experiments on single cells to relate captured genomic information obtained from the cell to functional and imaging analysis performed on the same cell.

### DESCRIPTION OF THE RELATED ART

Despite advances in research and technology of single-cell analysis, there continues to be a gap between attributing functional cellular data and molecular cellular data to cells at very high throughput. Conventional single cell analysis provides information on genetic profiles of cells and some select proteins, but not their functional state. Relating the functional characteristics of individual cells to their underlying molecular constitution, therefore, is incredibly valuable to understand, model and engineer biology. Techniques to render the association between molecular and functional states of cells, at scale, are challenging because of the diversity of analytes that need to be profiled as well as the difficulty in tracing spatially defined functional assays to pooled sequencing readouts on current versions of sequencers. In conventional single-cell sequencing approaches, genomic content from individual cells are pooled together before loading on a next-generation DNA sequencer. The molecular state of individual cells may be resolved through DNA barcodes that track nucleic acid content derived from individual cells. These barcodes, however, do not track the positional or temporal information of assays or perturbations these cells may have undergone, rendering it impossible to assign functional characteristics to the molecular profiles compiled by such single-cell sequencing methods.

Human tissues are comprised of highly heterogeneous mixture of cells interacting and regulating each other. As such, a comprehensive understanding of individual cells, their molecular signatures and their environment have vast applications in the fields of molecular biology and immunology, analytical chemistry and biochemistry, engineering, and, in particular, development of novel pharmaceutical compounds. In addition, cellular function is the foundational cornerstone in the understanding of the precise biology of disease states. As a result of the recent advances in single-cell analysis technology, understanding of genomes, transcriptomes and proteomes has increased. Consequently, single cell analysis is one of the fastest growing segments of genomics coinciding with transformative new methods to profile genetic, epigenetic, spatial, proteomic and lineage information in individual cells (Stuart, T., et al., 2019, Integrative single-cell analysis. Nat Rev Genet 20, pp. 257-272.).

High-throughput single cell multi-omics have been bolstered by innovations in a myriad of differing technologies. Many assays have been developed for ascertaining high quality functional data about individual cells. For example, these include, by way of example only, imaging, flow cytometry, and mass spectrometry. In addition, techniques including RNA-seq, In Situ, qPCR and microarrays have further propelled the explosion in single cell analysis and resulted in a wealth of novel genomic data. While these technological advances have allowed biologists, chemists, and biomedical researchers to decode whole genomes, a genomic sequence alone does not provide adequate cellular information to elucidate the functional relevance of gene products within a complex living system.

Advances in single-cell sequencing and molecular profiling have been enabled by advances in microfluidic manipulation of cells. In conventional approaches, single cells are isolated into water-in-oil droplets alongside DNA-barcoded beads, such that the cells are lysed upon being encapsulated within the droplets, and selected analytes are captured on the barcoded beads. By sequencing the barcodes on the beads and the nucleic acids captured therein, molecular states of single cells can be ascertained. In these types of approaches, the functional state of cells is not measurable either due to their rapid lysis and/or due to the lack of a priori knowledge of the bead-bound DNA barcodes. If the sequence of DNA barcodes on various beads was known, and assays were to be performed on cells confined with such beads prior to lysis, the functional assays may relate to the sequencing readouts.

Whereas staggering technological advances in the field have enhanced current knowledge, such techniques to map millions of DNA barcodes and to perform assays in confined volumes (prior to lysis) are incredibly complex and expensive. When integrated and combined, data gathered from functional analysis and genomic analysis render a more complete picture of cell state, various aspects of cellular relationships and interactions, as well as disease states. Thus, there remains a need for a cost-effective and efficient method to systematically couple the genomic information of a cell with its functional information obtained from various assays at high throughput.

### SUMMARY OF THE INVENTION

In one aspect, there is disclosed a method for deterministic single-cell loading in nanowells or examination areas with full access to culturing conditions and assays and methods to analyze cells in a mixture of plurality of cells are described in this disclosure. In another aspect, there is provided a method of using multi-indexed barcodes and capture beads to tether functional cellular data with its corresponding genomic sequence at very-high throughput.

The method for analyzing a cell or cells in a mixture of plurality of cells comprises providing a plurality of examination areas wherein at least one examination area includes at least one cell to be analyzed. Further, at least one of a plurality of multi-indexed barcodes, and at least one of a plurality of capture beads, wherein each capture bead further comprises a plurality of oligonucleotides are employed and, then, performing an assay in at least one of the plurality of examination areas. Assay information is then collected from the examination area by performing release-and-capture in the at least one examination area, wherein the release-and-capture comprises releasing nucleic acid from the cell into the examination area such that at least one of the released nucleic acids is captured by the plurality of oligonucleotides attached to at least one of the capture beads and releasing at least one of the indices from the multi-indexed barcode in the examination area such that the released index is captured by at least one oligonucleotide attached to the capture bead positioned near the examination area. At least one capture bead is then analyzed, wherein the analysis comprises identifying at least one nucleic acid captured from the cell and identifying at least one captured index from the multi-index barcode. A molecular-functional profile of the cell is then generated in the examination area, wherein each of the multi-indexed barcodes includes at least two indices and at least one of the plurality of oligonucleotides on each capture bead attaches to at least one nucleic acid fragment released from the cell in the examination area and to at least one index from at least one of the plurality of multi-indexed barcodes.

According to one aspect, the indices of the multi-index barcode comprise an optical index, an oligonucleotide index, a mass index, a charge index, a size index, a fluorescence index, a chemical index, a shape index, a hardness index, an ionization index, a nucleic acid index, a smell index and/or an audio index. The multiple indices of the multi-index barcodes may be bound to a common substrate, wherein the substrate comprises a solid substrate, a gel substrate, a dissolvable substrate, a crosslinked substrate, a nucleotide substrate, a chemical substrate, a droplet substrate, a biological substrate and/or a physical substrate.

In another aspect, there is provided a method for analyzing a plurality of capture beads. The method comprises exposing the capture beads to a plurality of cells, wherein at least one of the plurality of cells is in at least one of a plurality of examination areas. The capture beads are collected wherein each capture bead further comprises at least one of a perturbation agent, a plurality of capture oligonucleotides, genomic content captured from the cells exposed to the bead in the examination area, a multi-index barcode, a spatial-index barcode, and/or a plurality of non-nucleotide tags. The genetic content on the capture bead is sequenced, and the identity associated with at least one capture bead is generated, wherein the generated identity includes the sequence of the genetic content captured from at least one cell and the capture oligonucleotide, and wherein sequencing the capture bead includes sequencing the capture oligonucleotide and the genomic content of the capture beads, and wherein the sequence of the genetic content includes nucleic acid captured from cells exposed to the capture beads, and the generated identity further comprises synthesis history of the perturbation agent on the bead and/or spatial location of the cells exposed to the beads.

According to one aspect, the plurality of perturbations represented by the perturbation agents comprises genetic perturbations, chemical perturbations, peptide perturbations, protein perturbations, cellular perturbations, antigen perturbations, antibody perturbations, perturbations generated by physical stress, environmental stress including temperature, pH, exposure time, and/or solvent perturbations.

According to another aspect, identifying the location of the examination area comprises contacting the examination area with at least one of the plurality of beads, wherein the bead is identifiable by a captured spatial index.

Also provided herein is a method for single-cell barcoding using non-distinguishable beads, the method comprising: introducing at least one non-distinguishable bead to an examination area, wherein the examination area has at least one cell of interest; liberating nucleic acid content from the cell of interest into the examination area; capturing the nucleic acid content on the non-distinguishable bead; extracting the non-distinguishable bead from the examination area; and introducing at least one barcode to the non-distinguishable bead, wherein the barcode is attached to the nucleic acid content captured on the non-distinguishable bead and the barcode is unique to the non-distinguishable bead.

Further disclosed is a method for single-cell barcoding comprising: providing at least one examination area, the examination area including at least one cell to be analyzed, and at least one non-distinguishable capture bead; lysing the cell in the examination area to extract nucleic acid content from the cell; capturing at least a portion of the nucleic acid content on the non-distinguishable capture bead; extracting the non-distinguishable capture bead from the examination area; and synthesizing at least one unique barcode, wherein the unique barcode is introduced to the end of the captured nucleic acid content on the non-distinguishable capture bead.

Also provided herein is a composition comprising a non-distinguishable solid support and a synthesized barcode, the synthesized barcode further comprising a degenerate and/or indistinguishable capture sequence and captured nucleic acid content from a lysed cell, wherein the nucleic acid content is coupled to the degenerate and/or indistinguishable capture sequence.

Further provided herein is a method for barcoding a non-distinguishable bead comprising: exposing the non-distinguishable bead to a cell of interest; lysing the cell of interest; capturing nucleic acid content from the cell of interest on the non-distinguishable bead, wherein the nucleic acid content is captured on the non-distinguishable bead via at least one degenerate and/or indistinguishable capture sequence.

Also disclosed herein is a method for capturing positional information from single-cells using a solid support having an optical barcode comprising: providing at least one examination area, the examination area including at least one cell to be analyzed, and at least one capture bead, the capture bead having at least one optically distinguishable barcode, wherein the optical barcode corresponds to the examination area; lysing the cell in the examination area, wherein lysing the cell releases nucleic acid content; capturing at least a portion of the nucleic acid content via the capture bead; extracting the capture bead from the examination area; performing ledger synthesis on the bead, wherein the ledger synthesis produces a unique nucleotide-based barcode that corresponds to the optical barcode.

Further provided herein is a method for corresponding spatial information to a single-cell using optically barcoded beads comprising: providing at least one examination area, wherein the examination area has at least one cell; exposing the cell to an optically barcoded bead in the examination area, wherein the optical barcode of the bead corresponds to the examination area; releasing nucleic acid content from the cell, capturing the nucleic acid content on the optically barcoded bead; removing the optically barcoded bead from the examination area; conducting split-pool synthesis on the optically barcoded bead, such that a unique barcode is synthesized on the optically barcoded bead, wherein the unique barcode includes nucleotides; coupling the unique barcode and the optical barcode from the optically barcoded bead; and corresponding the unique barcode and the optical barcode to the examination area.

Further disclosed is a method for performing spatial analysis on a single cell using ledger synthesis comprising: placing at least one cell and at least one optically barcoded bead in an examination area; extracting nucleic acid content from the cell; capturing the nucleic acid content with the optically barcoded bead; removing the optically barcoded bead from the examination area; performing split-pool synthesis, wherein a unique barcode is generated and coupled to the optically barcoded bead; tracking the optical barcode using a ledger; and sequencing the optically barcoded bead, wherein the unique barcode and the optical barcode correspond to the examination area.

Further provided herein is a composition comprising a solid support having an optical barcode, the solid support further comprising nucleic acid content of a lysed cell and a plurality of unique nucleotide fragments, wherein the plurality of unique nucleotide fragments forms a unique barcode, and the unique barcode corresponds to the optical barcode of the solid support.

### BRIEF DESCRIPTION OF THE DRAWINGS

FIG. 1A illustrates cells isolated in a planar examination area;

FIG. 1B is an example of droplets as examination areas, wherein the droplets are isolated from each other within an immiscible medium;

FIG. 1C is an example of an isolated microwell array as an examination area;

FIG. 2A is an example of an isolated microwell array as an examination area that is confined and isolated;

FIG. 2B is an example of a fluidically connected examination area.

FIG. 2C illustrates an example of an examination area with some isolated examination areas and some fluidically connected examination areas;

FIG. 2D illustrates a three dimensional (3D) example of an examination chip that contains multiple examination areas in the form of arrays of nanowells wherein the top surface (or the openings) of the nanowells are planar with the top surface of the examination chip;

FIG. 2E illustrates a 3D example of an examination chip wherein the top surface of the examination areas (nanowell arrays) are not coplanar with the topmost surface of the examination chip. In this illustration the nanowell arrays are recessed from the top surface of the examination chip such that a fluidic chamber is created above the top surface of the examination areas allowing fluidic connection between the examination areas;

FIG. 3A is an example of a single-index barcode bead, the singular detection method is an optical index;

FIG. 3B is an example of a single-index barcode bead, the singular detection method is an oligonucleotide index;

FIG. 3C is an example of a single-index barcode bead, the singular detection method is a mass index;

FIG. 4A is an example of a multi-index barcode bead having optical and mass indices;

FIG. 4B is an example of a multi-index barcode bead having oligonucleotide and mass indices;

FIG. 4C is an example of a multi-index barcode bead having optical and oligonucleotide indices;

FIG. 4D is an example of a multi-index barcode bead having optical, oligonucleotide, and mass indices;

FIG. 5A is an example of a capture bead presenting antigen or aptamers to capture antibodies or proteins;

FIG. 5B is an example of a capture bead presenting antibodies to capture proteins;

FIG. 5C is an example of a nucleic acid capture bead with oligonucleotide barcodes;

FIG. 5D is an example of a capture bead with nucleic acid captured;

FIG. 6A is an example of a bead having chemical perturbations;

FIG. 6B is an example of a bead having nucleic perturbations;

FIG. 7 is an example of a generic multi-index, multi-functional bead having detectable indices, perturbations, and capture elements. The various indices of the bead may include oligonucleotide capture elements, oligonucleotide or perturbations, mass tags that encode the beads or the perturbations on the bead, genetic perturbations, chemical perturbations, and optical tags that identify the bead or the perturbations or capture elements on the bead;

FIG. 8A illustrates an example of multi-index (MI) beads in contact with cells on a planar examination array;

FIG. 8B illustrates an example of MI beads disposed inside droplets;

FIG. 8C illustrates examples of nanowells with MI beads inside the nanowells, MI beads suspended in a separate layer on top of the isolated nanowells, MI beads suspended on top of the nanowells and MI beads suspended on top of fluidically connected nanowells;

FIG. 9A shows an example of a nanowell examination area, wherein the examination area has cells and one or more positional index MI beads, and a capture bead is above the examination area;

FIG. 9B shows a nanowell examination area with MI barcodes including a DNA index and optical index and a capture bead above the examination area;

FIG. 10 illustrates an example of cells delivered to a microwell array by gravity sedimentation;

FIG. 11 illustrates an example of cells and MI barcode beads dispersed into the examination area. Attached to the cells are optical labels and attached to the MI beads are nucleotide barcodes where the nucleotide barcodes are releasable by thermal denaturation or photocleavage;

FIG. 12A illustrates an example of uniquely identifying individual wells of a nanowell array using optical barcodes on multi-index beads. The optical barcodes of the multi-index barcode beads are deposited into the nanowell examination areas and decoded, here each bead may be one of 20 possible beads, where the 20 bead barcodes are identifiable by the ratio of two colors;

FIG. 12B is an example of capture beads disposed into examination areas that contain cells and multi-index barcode beads including optical tags that identify the oligonucleotide tags that are released from the multi-index beads and captured by capture beads;

FIG. 13A is an example of the top surface of a lid that is used to create a reversibly enclosable examination chip. The top surface contains a pliant silicone gasket that will contact the top portion of a clamp that will be screwed to the bottom portion of the clamp. The pliant material is placed atop a clear optical window to allow light passage and optical interrogation of the chip enclosed by the clamp. The lid also comprises holes that allow fluidic connection to the examination areas enclosed by the reversibly enclosable clamp;

FIG. 13B is an example of the bottom surface of a lid that is used to create a reversibly enclosable examination chip. The bottom surface of this lid presses against an examination chip, which is then pressed against the bottom portion of a clamp;

FIG. 14A illustrates the closed form of the clamp containing an examination chip sandwiched between a lid with optical windows and the top and bottom portions of the clamp;

FIG. 14B illustrates an exploded view of the clamping architecture showing how the examination chip sits under a lid which are then clamped together by the top and bottom portion of the clamp;

FIG. 15A illustrates an example of single-cell barcoding using non-distinguishable beads. Briefly, a non-distinguishable bead is placed in an examination area with a cell of interest, upon lysing, nucleic acid content is released from the cell and captured by the non-distinguishable bead and beads are extracted from the examination area;

FIG. 15B shows the process of synthesizing a bead-unique barcode using split-pool oligonucleotide extension;

FIG. 16A shows examples of optically barcoded beads. Ledger synthesis correlates the optically barcoded beads to a spatial position;

FIG. 16B illustrates an example of ledger synthesis after optical imaging of a first split: the Orange bead is appended with barcode fragment I, the Teal bead is appended with barcode fragment II, and the Blue bead is appended by barcode fragment III; and

FIG. 16C illustrates an example of ledger synthesis after optical imaging of a second split: the Orange bead is appended with barcode fragment II, the Teal bead is appended with barcode fragment III, and the Blue bead is being appended by barcode fragment I.

### DETAILED DESCRIPTION OF THE EMBODIMENTS

Disclose are methods and devices for conducting cellular analysis at high throughput which comprise capturing and culturing cells in isolated or fluidically connected examination areas. In the examination area, experiments may be performed on the cells or on clonal copies of the cells. Cells in the examination area may be imaged, and genomic content and/or other material from the lysed cells may be captured using single-index or multi-index capture beads. The capture beads may correlate the captured genomic content from cells to the examination area where functional experiments were performed on the same cells. As a result, captured genomic information and experimental functional data as well as imaging may be spatially indexed, allowing for an integrated high throughput system that may analyze a single cell or population of cells. In addition, capture beads may have a single index or multiple indices and/or perturbations. The perturbations may include, for example, genetic perturbations, chemical perturbations, peptide perturbations, protein perturbations, cellular perturbations, antigen perturbations, antibody perturbations, perturbations generated by physical stress, environmental stress including temperature, pH, exposure time and/or solvent perturbations.

### Definitions

The terminology used herein is for the purpose of describing particular embodiments only and is not intended to be limiting of or to the disclosure. As used herein, the singular forms “a”, “an” and “the” are intended to include the plural forms as well, unless the context clearly indicates otherwise.

The term “about” when used before a numerical designation, e.g., temperature, time, amount, concentration, and such other, including a range, indicates approximations that may vary by (+) or (−) 10%, 5%, 1%, or any subrange or a subvalue there between. In one embodiment, the term “about” when used with regard to a dose amount means that the dose may vary by +/−10%.

The term “comprising” or “comprises” is intended to mean that the compositions and methods include the recited elements, but not excluding others.

The term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure.

The term “consisting of” shall mean excluding more than trace elements of other ingredients and substantial method steps. Embodiments defined by each of these transition terms are within the scope of this disclosure.

The term “assay” is meant the determination of the amount of a particular constituent of a mixture or of the biological or pharmacological potency of a drug. For purposes of this disclosure, assay may comprise an imaging assay, a morphology assay, a cell-painting assay, a luminescence assay, a fluorescence assay, a thermal shift assay, a nucleic acid hybridization assay, a FISH assay, a cytokine release assay, an antibody release assay, an antibody detection assay, an immunofluorescence assay, a cellular co-culture assay, a cellular growth assay, a cytotoxicity assay, a proliferation assay, a cell killing assay, a protein degradation assay, an enzyme inhibition assay, a protein-protein interaction assay, a protein-small molecule interaction assay, and/or a protein-nucleic acid interaction assay

By “bead” is meant any material capable of occupying finite space in the absence of confinement and capable of serving as an attachment substrate to various analytes of this disclosure. For purposes of this disclosure the bead may comprise shapes that are not spherical or regular, for instance comprising gels, matrices, semiconductor chips, metallic particles including magnetic metallic particles, or polymeric shells.

By “capture bead” is meant a bead capable of capturing one or more of various materials the bead is sensitized to capture. For purposes of this disclosure, a capture bead may be capable of capturing one or more of nucleic acids, peptides, proteins, metabolites, sugars, carbohydrates, lipids, cholesterol, nanoparticles, organic compounds, inorganic compounds, synthetic polymers, natural polymers, biopolymers, entire cells, or components of cells.

By “cell multiplication” is meant a cell division or cell culturing or cell proliferation, which is a process of clonal expansion of a single cell in to multiple copies of the single cell, wherein the multiple copies are generated by the process of mitosis or meiosis or another mechanism of cell division and proliferation.

By “examination area” is meant a place in which cells are disposed. An examination area can also equivalently refer to the smallest area in which a cell may be present or an assay may be performed, such that the examination area for an experiment may comprise a plurality of examination areas each capable of examining one or more of a plurality of cells, wherein the examining represents the conventional meaning of the term implying observation, visualization or analyzing. In various embodiments of this disclosure the examination area may comprise a solid substrate, a hydrogel substrate, a liquid droplet, a plurality of liquid droplets, a hydrogel droplet, a plurality of hydrogel droplets, a microwell or a microwell array, a nanowell or a nanowell array, a picowell or a picowell array, a glass substrate, a plastic substrate, a photoresist substrate, a smooth substrate, a perforated substrate, a micro-patterned substrate etc.

By “examination chip” is meant a surface that contains one or more examination areas. The examination chip may refer to configurations of examination areas that may optionally and/or reversibly be enclosed within chambers with fluidic access. The examination chip may also equivalently be referred to as an assay substrate. It is to be noted that the examination chips of this disclosure refer to a higher-order structures that comprises examination areas, and as such the examination areas may be present in recessed or raised or otherwise modified areas of the examination chips. More specifically the top surfaces of the examination areas need not correspond to the top surfaces of examination chips.

By “fluidically connected” is meant a fluidic path between examination areas such that materials miscible in fluids comprising the fluidic connection are exposed to all the connected examination areas. For the purposes of this disclosure, the fluids comprising the fluidic connection may or may not be identical to the fluids contained within the examination areas being connected by the fluidic connection, whereas in some embodiments the fluidic connection is made between fluids contained within the connected examination areas.

By “molecular-functional profile” of a cell is meant a cell state represented by one or more molecular constituents of the cell and one or more functional characteristics of the cell, wherein the functional characteristics may be a manifestation of the sum total of interactions between various molecular constituents and environment of the cell. The molecular constituents of the cell may comprise any one or a collection of analytes contained in the cell, on the cell or secreted by the cell. The same assay modality may be used to study both functional and molecular states of cells. For instance, imaging assays may be used to profile both molecular and functional states of cells based on the feature and characteristics being imaged.

By “multi-omic analysis” is meant analysis of multiple distinct molecular signatures of the same cells. Such molecular signatures of a cell may comprise the cells genetic composition, nucleic acid composition, proteomic composition, metabolite composition, genetic architecture and 3D organization, lipid composition, and the like, wherein multi-omic analysis suggests collecting information on multiple such molecular signatures from the same cell, optionally for multiple cells.

By “indices” is meant orthogonal tags. The terms “indices” may be used interchangeably with “index” or “barcode.” Indices may include but are not limited to optical barcodes, oligonucleotide barcodes, mass barcodes, electrical barcodes, radio-frequency barcodes, and/or any other detectable barcodes.

By “linkers” is meant cleavable and/or releasable linkers, or non-cleavable linkers, such that the perturbations may be released from the beads by cleaving the linker. For example, a cleavable linker may comprise a photocleavable linker such as a nitro-benzyl containing linker, or any other suitable photocleavable linker, a thermal cleavable linker, an enzyme cleavable linker, a chemical cleavable linker and/or any other suitable cleavable linker. Further, the non-covalent interaction of biotin-avidin may also serve as a linker.

By “library of perturbations” is meant a collection of one or more agents which when exposed to cells may elicit a molecular or functional response from the cells. In contrast, in some examples, none of the perturbation agents in the library of perturbations may elicit a response from the cells. The terms “perturbagens”, “perturbation agents” and “perturbations” may be used interchangeably with the term “library of perturbations”.

By perturbation or perturbation agents is meant materials that are intended to elicit a molecular or functional response from one or more cells. In contrast, in some examples perturbation or perturbation agents may not elicit a response from any cells.

By “synthesis history” is meant the set of steps implemented to synthesize a perturbagen of a perturbation library.

By “multi-index barcode beads” is meant beads that are identifiable by multiple orthogonal tags (barcodes).

By “ledger-synthesis” is meant a method to track information about beads in a bead-information ledger such that any observable property of beads may be used to track any processing steps performed on the bead by appending such information on the bead-information ledger. Processing steps on beads tracked on the bead-information ledger may include chemical synthesis on beads, optical encoding on beads, oligonucleotide synthesis on beads, oligonucleotide barcode synthesis on beads, mechanical manipulation of the beads, imaging of the beads, assays performed on the beads, analytes captured on the beads or any process involving modifying the beads in any way.

By “bead-information ledger” or “ledger” is meant a record or register of information on various beads involved in an experiment. The ledger may be an electronic document such as a computer file, a computer program, a cryptographically encoded distributed ledger, a physical document, or any method capable of tracking information on beads. It is to be understood that recording information about a bead may be different from encoding information on a bead; encoding representing a one-to-one correspondence between a constituent of a barcode and a constituent of an entity attached to a bead, whereas recording, for instance recording information on a bead-information ledger does not represent a one-to-one correspondence but rather a tracking mechanism for constituents on a bead. For illustration purposes, a bead containing optical barcodes [red, green, blue] may only encode a singular entity [A,B,C] where the color red encodes the entity A, the color green encodes the entity B and blue encodes the entity C. A bead with barcodes [red, blue] would encode the entity [A,C] and the bead with barcodes [green, blue] would encode entities [B,C] in this example. In contrast, a bead barcoded [red, green, blue] may record any information about the bead on a ledger. For instance, the bead [red, green, blue] may record entities [C,A,C], while the bead [red, blue] may record entities [X, Y], and the bead [green, blue] may record entities [L, A] on the ledger. (The entities A, B, C, X, Y, L of this example may be chemical building blocks, oligonucleotide barcodes, mass barcodes, any barcodes, any material that is attached to the bead, or any property of the bead].

By “off-chip experiments” it is meant experiments that are conducted outside of the examination areas on material that may have been present in examination areas. Such materials may comprise cells or molecular constituents of cells or beads or other analytes that were originally present in examination areas and in some embodiments subjected to experiments or assays within the examination areas.

By “on-chip experiments” it is meant analysis, assays, observations or experiments conducted on materials present within or proximal to examination areas. Such materials may comprise cells or molecular constituents of cells or beads or other analytes present within or proximal to examination areas.

By “release-and-capture” is meant liberation of material from one entity present within or proximal to an examination area and subsequent capture of a fraction, whole or none of the liberated material by another or the same entity present within or proximal to the examination area. Said entities may comprise various elements of the disclosure, such as cells, beads, multi-index beads, capture beads or other entities contemplated as being present within or proximal to the examination areas of this disclosure.

Current technology allows researchers to analyze single cells in remarkable detail. Genomic detail may be ascertained from single cells using a variety of “off-chip” techniques. Next generation sequencing, specifically RNAseq, allows for the construction of the cellular transcriptome, providing insight into functional elements of the genome and revealing the molecular constituents of cells and tissues. In addition, functional assays and imaging assays also proffer a significant amount of information pertaining to a single cell. When integrated, data gathered from genomic experiments coupled with data gathered from functional and imaging assays may advance understanding of cellular interactions with its surrounding environment, disease development and therefore allow for enhanced treatments and therapies.

Described herein are systems and methods of a multi-functional, multi-omic platform that integrates cellular genomics, functional assays, visualization or imaging and the addition of perturbations at high throughput. Further, described herein are methods that effectively couple off-chip experiments (for example capture RNA/DNA/cytokines) and on-chip experiments (for example, imaging, any suitable assay) by indexing the activities via single or multi-index beads. Accordingly, the present invention provides systems and methods for trivially and robustly combining cellular perturbations, functional experimental data and genomic analysis at very high throughput. Also disclosed, are methods to deploy assay and/or spatial barcodes corresponding to a cell or a population of cells that may be efficiently carried over to sequencing so that data obtained via functional experiments may be combined with sequencing data to efficiently identify a single cell. The terms index, indices, and barcodes may be used interchangeably throughout. The index or barcodes may be positional and/or employ a unique poly-T tail. The indices and/or barcodes associated with the beads may or may not capture mRNA or other genomic information from cells in a given examination area. Each individual examination area may be assigned a unique spatial barcode to associate with the cells in the same examination area.

Described herein are methods of analyzing cells, the cells may comprise eukaryotic and/or prokaryotic cells, or a combination of eukaryotic cells and prokaryotic cells. A cell may be any biological material confined within a membrane that is capable of replicating itself under appropriate conditions. In certain embodiments of the disclosure the cells may be fixed and/or denatured cells. In addition, cells may be reporter cells.

Examples may provide for a plurality of cells comprising a homogeneous or a heterogenous mixture of cells including mammalian cells, eukaryotic cells, bacterial cells, fungal cells, plant cells, prokaryotic cells, immune cells, cancer cells, antibody secreting cells, genetically modified cells and/or cells of aliens from outer space and new worlds.

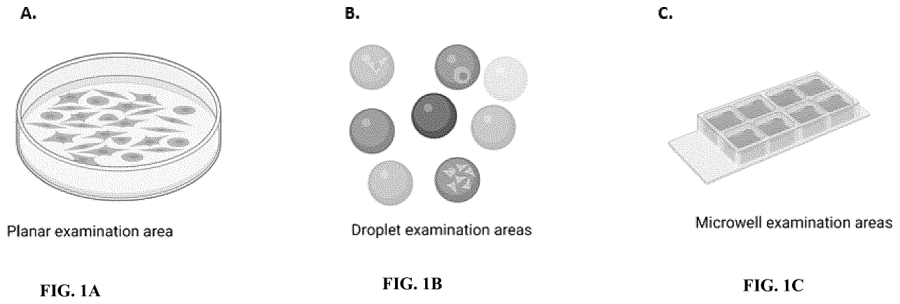

Referring to FIGS. 1A-1C, a plurality of examination areas may be isolated by varying methods. For example, FIGS. 1A-1C provides examination areas that may include droplets, surfaces, microwells, nanowells, picowells, beads, surfaces of beads, glass slides, hydrogel matrices and, in non-limiting terms, other areas in which a cell may be placed. The plurality of examination areas may be isolated. For example, examination areas may be isolated using fluidic isolation, mechanical isolation, physical isolation, chemical isolation, thermal isolation, optical isolation and/or genetic isolation. FIGS. 1A-1C also show examples of droplet isolation, bead isolation, microwell isolation, nanowell isolation, picowell isolation. Other forms of isolation may be contemplated, including isolation in aqueous media surrounded by oil media, isolation in confined physical geometries comprising aqueous media capped by mechanical, and/or providing oil or air barriers.

Generally speaking, cells may be placed in an array of examination areas, the array of examination areas may be interchangeably referred to as the examination area or assay substrate, and the examination areas may be present on examination chips. Broadly, cells are confined in, and analyzed in, or near, the examination areas. The cells may be disposed in examination areas such that single cells are present in each examination area. In some embodiments the examination areas are arrays of microwells. Detailed descriptions of suitable dimensions and materials for arrays of microwells are provided in U.S. patent. Nos. U.S. Ser. No. 10/828,643B2, U.S. Ser. No. 11/027,272B1, US2021/0229087A1, and U.S.2021/0308668A1 incorporated herein by reference. In some embodiments the examination areas are microwell arrays where some or all of the microwells have one or more pores in the bottom such that pores act as a fluidic conduit across the microwells. The dimension of the pores at the bottom of the microwells may be any fraction less than one compared to the dimensions of the bottom of the microwells. For instance, the size of the pore may be 0.001%, 0.01%, 0.1%, 1% 10% or 90% of the dimension of the bottom of the microwell. In some aspect's cells may be trapped in examination areas by a negative pressure such as a vacuum being applied from the examination area (Abali, F., 2016, Expansion of Cancer Cells in Self-Sorting Microwells, Medical Cell Biophysics Group, University of Twente) (for example, U.S. Pat. Nos. US2021/0229087A1 and US2020/0243290A1).

Alternatively, or in addition, cells may be pushed into examination areas by positive pressure such as hydrostatic pressure. Single cells may be trapped in single examination areas.

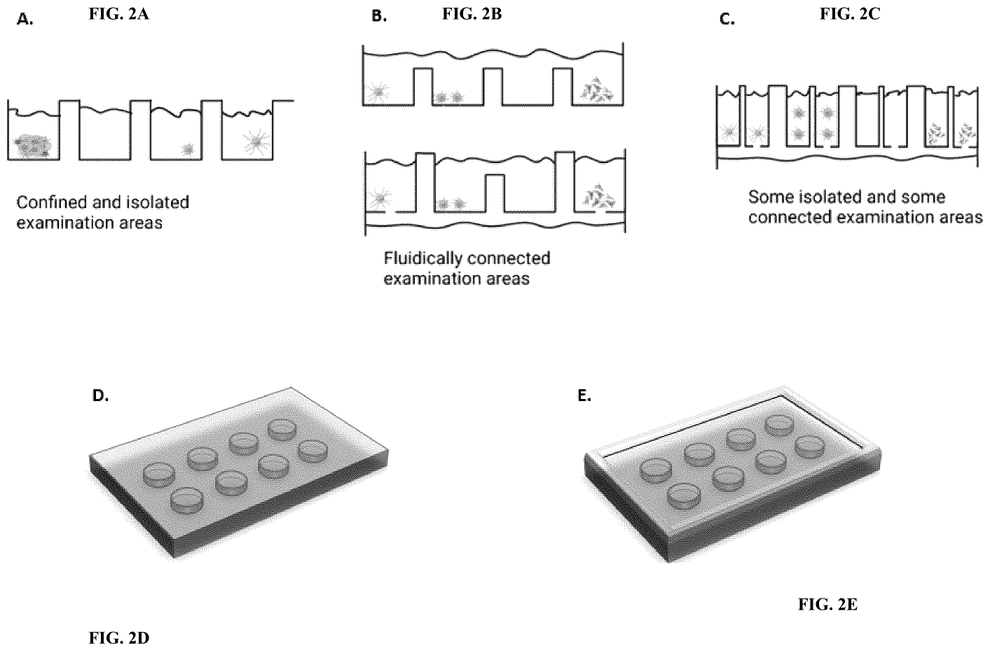

The examination areas may be isolated from one another. For example, FIG. 1A provides an example of confined and isolated examination areas, whereas FIG. 2B shows examination areas in fluidic contact with one another. Another example as shown in FIG. 2C includes isolated portions of the examination areas whereas a different portion of the examination areas may be in fluidic contact with one another. In addition, one of the plurality of examination areas may be a split examination area and further the split examination area may itself be split into multiple examination areas. In addition, FIGS. 2D and 2E provide different versions of an examination chip that contains multiple examination areas in the form of arrays of nanowells. As shown in FIG. 2D, in some examples the top surface (or the openings) of the nanowells are planar with the top surface of the examination chip. In some embodiments, the top surface of the examination areas (nanowell arrays) are not coplanar with the topmost surface of the examination chip. For example, FIG. 2E shows recessed nanowell arrays, such that a fluidic chamber is created above the top surface of the examination areas and thus allowing fluidic connection between the examination areas.

Methods to make droplet examination areas for cells are well known in literature (for example, Betterelli Giuliano, Microfluidic Reviews, 2020; Matula, et al. Adv. Biosystems, 2019, doi.org/10.1002/adbi.201900188). Methods to make microwell, nanowell and picowell arrays are also well known in literature (for example, Kim, et al. Biomed Eng Lett, 2013, 3:131-137; Lindström and Andersson-Svahn, Biochimica et Biophysica Acta 1810, 2011, 308-316; Manzoor et al. Can. J. Chem. Eng., 2021, 99:61-96).

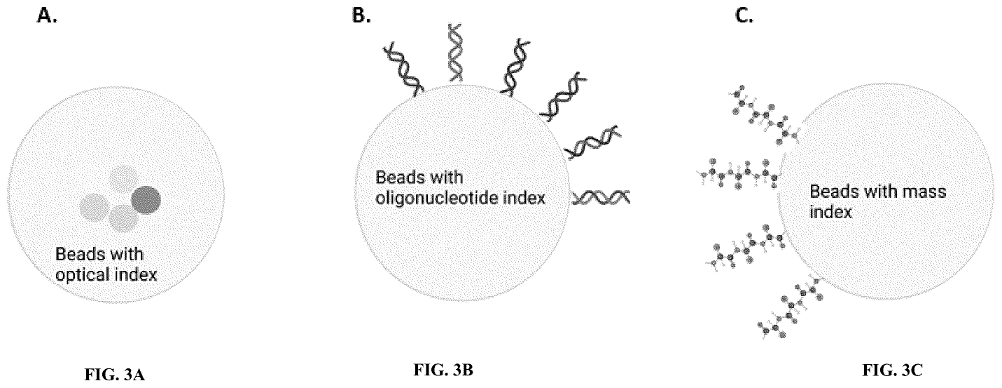

FIG. 3A-C provide examples of single-index barcode beads also referred to as capture beads. Briefly, beads may be linked to one or more index or indices. Such index may, for example, include an optical index, an oligonucleotide index, a mass index, a charge index, a size index, a fluorescence index, a chemical index, a shape index, a hardness index, an ionization index, a nucleic acid index, a smell index/or an audio index.

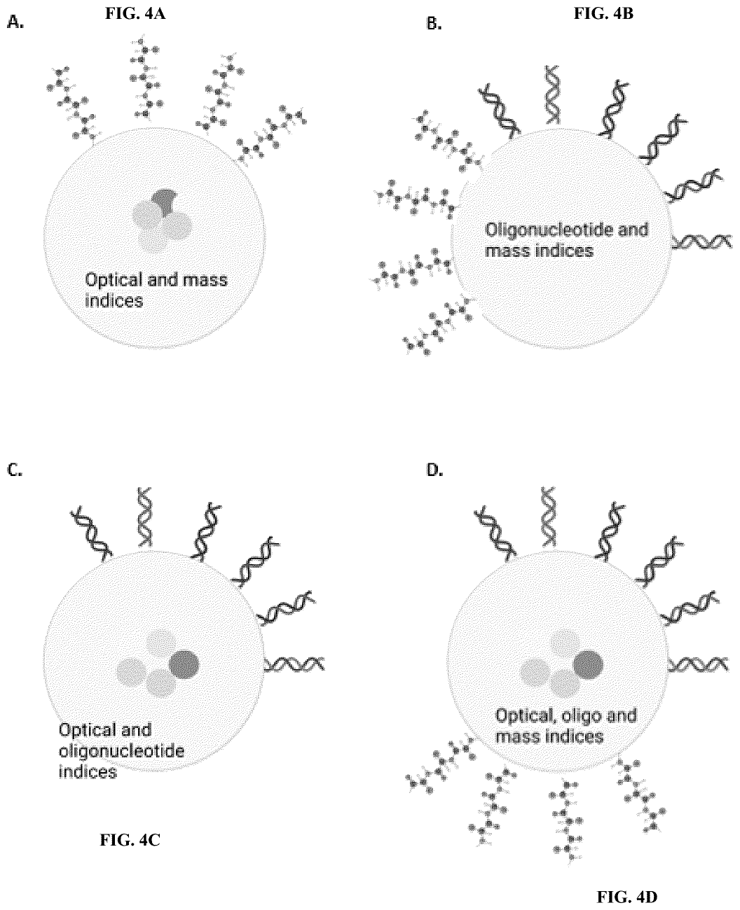

As seen in FIG. 4A-D, beads may also comprise multi-index beads. For the purposes of this disclosure beads that are identifiable by multiple orthogonal tags (barcodes) are called multi-index barcode beads (wherein the orthogonal tags are called indices for the purposes of this disclosure). The multi-index barcode beads may comprise oligonucleotide barcodes and/or optical barcodes. Alternatively, or in addition, the multi-index barcode beads may comprise oligonucleotide barcodes and mass barcodes. In yet other examples, the multiple indices may comprise mass barcodes and optical barcodes. In general embodiments the indices may be selected from one or more of optical barcodes, oligonucleotide barcodes, mass barcodes, electrical barcodes, radio-frequency barcodes, or other detectable barcodes. Attaching the various indices to beads may be accomplished by various ways commonly known to practitioners in the field. A review of functionalizing porous beads may be found (Gokmen & Du Prez, Progress in Polymer Science, 2012, 37, 365-405.)

An example of multi-index beads includes multiple indices of the multi-index barcodes bound to a common substrate. For example, the substrate may include a solid substrate, a gel substrate, a dissolvable substrate, a crosslinked substrate, a nucleotide substrate, a chemical substrate, a droplet substrate, a biological substrate and/or a physical substrate.

Another example of multi-index beads may include at least one index that uniquely identifies the bead from at least one other bead in a population of multi-index barcode beads. In addition, a combination of indices on a bead may uniquely identify the bead from at least one other bead in a population of multi-index barcode beads. For example, at least one index on a multi-index barcode bead may identify one or more of the other orthogonal indices on the multi-index barcode bead.

Alternatively, or in addition, the multi-index barcode beads may be the capture bead, a perturbation bead or a perturbation-encoded capture bead. Further, one or more of the multi-index barcode beads may be disposed within or in proximity to each examination area such that the multi-index barcode beads present within, or in proximity to, an examination area are capable of distinguishing one examination area from another examination area. In addition, multiple multi-index barcode beads may be disposed within different examination areas.

An example of a multi-index barcode bead may include one or more of the indices (a given index representing one type of barcodes) on a multi-index barcode bead being releasable from the multi-index barcode bead. The mechanism of release may comprise one or more of photo-release, thermal release, acoustic release, chemical release, or other release mechanism. The multiple indices on a multi-index barcode bead may be releasable by similar or different release mechanisms. Alternatively, or in addition, different indices on different multi-index barcode beads may be releasable by the same release mechanism. Multiple indices on a multi-index barcode bead may also be releasable by the same release mechanism.

Another example includes barcodes (or indices) that are released from a multi-index barcode bead that may be captured by another element in the examination area. The barcodes released by the multi-index barcode bead may also be captured by a capture bead disposed within or in proximity to an examination area. Further, one of the un-released indices on the multi-index capture bead may identify the released barcode captured by the capture bead. Alternatively, or in addition, capture beads may capture the barcodes released from one or more multi-index barcode beads alongside genomic content from cells in the examination area comprising, or close to, the capture bead. The multi-index barcode beads may additionally capture genomic content, proteomic content, metabolomic content or any other suitable analyte produces by cells. In other examples, the barcodes released from multi-index barcode beads may be capable of serving as perturbations to cells within examination areas proximal to the multi-index barcode beads. The multi-index barcode beads may also attach themselves to capture beads.

An example of the different indices on multi-index barcode beads may be an optical index and oligonucleotide barcodes and different multi-index barcode beads may comprise different optical barcodes and different nucleotide barcodes. For example, the optical barcodes present on a multi-index barcode may uniquely identify the oligonucleotide barcodes present on that multi-index barcode bead. Further, the oligonucleotide tags on the optical-oligonucleotide multi-indexed beads may be releasable from the bead and the released oligonucleotides may be capable of being captured by capture beads in proximity to the multi-index beads and the identity of the oligonucleotide captured by a capture bead may be identifiable by the identity of the optical barcode found on the multi-index barcode bead in proximity to the capture bead.

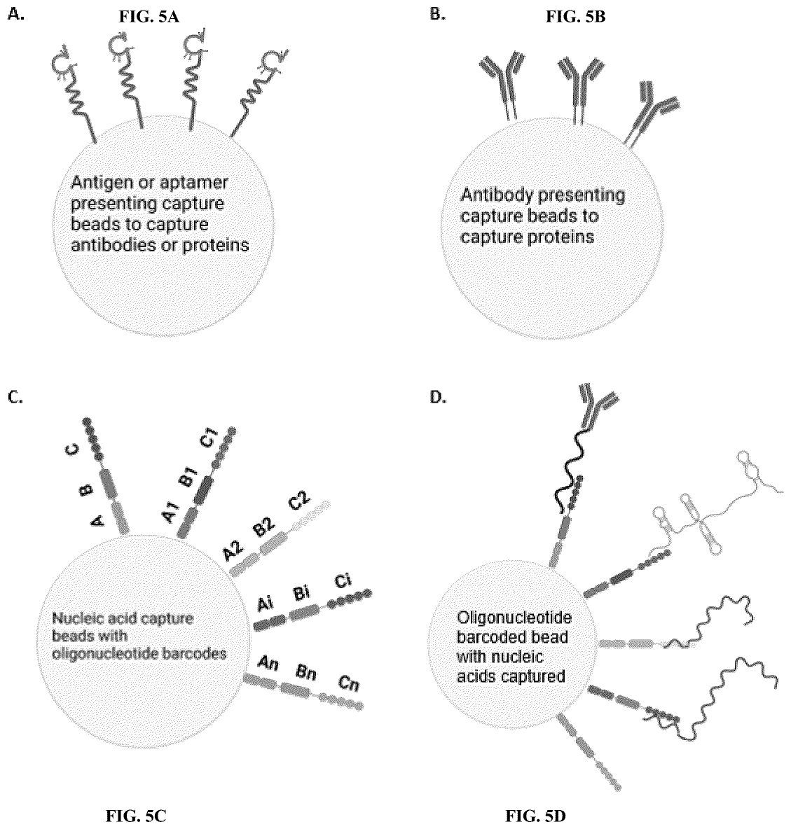

An example of the capture beads is shown in FIG. 5A-D. Capture beads may have barcode oligonucleotides and each capture bead may comprise a plurality of the oligonucleotide barcodes. The plurality of oligonucleotide barcodes on the bead may distinguish the bead from other beads in a population of beads (FIG. 5C). For example, a plurality of capture oligonucleotide on each bead may comprise a segment that can hybridize to complementary nucleic acid sequences of cells (FIG. 5D). The complementary sequence may be a poly-thymine (poly-T) sequence capable of hybridizing to 3′ poly-A sequences of mRNA molecules inside a cell. Alternatively, or in addition, the plurality of oligonucleotides on a capture bead may also encode the identity of any perturbation contained on the capture bead and in such instances the capture bead may also serves as the perturbation bead. The capture oligonucleotides may further capture the response of the cells to the perturbations delivered by the same bead. Various methods to generate oligonucleotide encoded capture beads are known in literature, as examples publications (Delley and Abate, Scientific Reports, 2021, 11:10857; Wang, et al., Adv. Sci., 2020, 7, 1903463; and Cheng, et al., Nature Portfolio, 2018, DOI:10.1038/protex.2018.116). The oligonucleotide barcodes may also encode or identify the perturbation present on the bead. A method for generating DNA encoded perturbation beads is found in (Price, at al. Anal. Chem. 2016, 88, 5, 2904-2911) publication, and approached to utilize DNA-encoded perturbation beads as capture beads are disclosed in U.S. Pat. Nos. US2019/0210018A1, US2021/0229087A1, US2021/0402404 A1, U.S. Ser. No. 11/084,037B2, U.S. Ser. No. 10/981,170B2, U.S. Pat. No. 9,638,636B2, U.S. Ser. No. 11/027,272B1 and U.S. Ser. No. 11/040,343B1.

Another example of capture beads may be encoded by oligonucleotides (FIG. 5C). Alternatively, or in addition, capture beads may be encoded by other means rather than by oligonucleotides. For example, the capture beads may be encoded by mass barcodes (FIG. 4D) and each capture bead may comprise a unique tag whose identity can be determined by measuring the mass of the tag, for instance in a mass spectrometer. The mass tags may additionally uniquely identify the bead from at least one other bead in a population of beads. One approach to create encoded beads capable of being identified by mass spectroscopy is provided in Nestler, at al., Anal. Chem. 2016, 88, 5, 2904-2911. A general method to identify mass encoded beads is described (Lee, at al., Anal Chem. 2010 Jan. 15; 82(2): 672-679.).

Another example of the capture beads comprises one or more optical tags, wherein the optical tags are capable of uniquely identifying the bead in a population of beads (as seen in FIG. 4C-D). The optical tags may be present on the surface of the beads. Alternatively, or in addition, the optical tags may be present in the interior of the beads. The optical tags may be, for example, fluorescence tags, scattering tags, optical absorption tags, luminescent tags, laser tags, photonic tags or plasmonic tags. A summary of various approaches to generate optically encoded beads is presented in (Lin, at al. Chem 4, 2018, 997-1021) review article. In some embodiments the optical tags encode perturbations present on the beads. Preparation methods and utility of optically encoded perturbation beads are described in Application PCT/US2020/048666 patent application, incorporated in its entirety herein by reference.

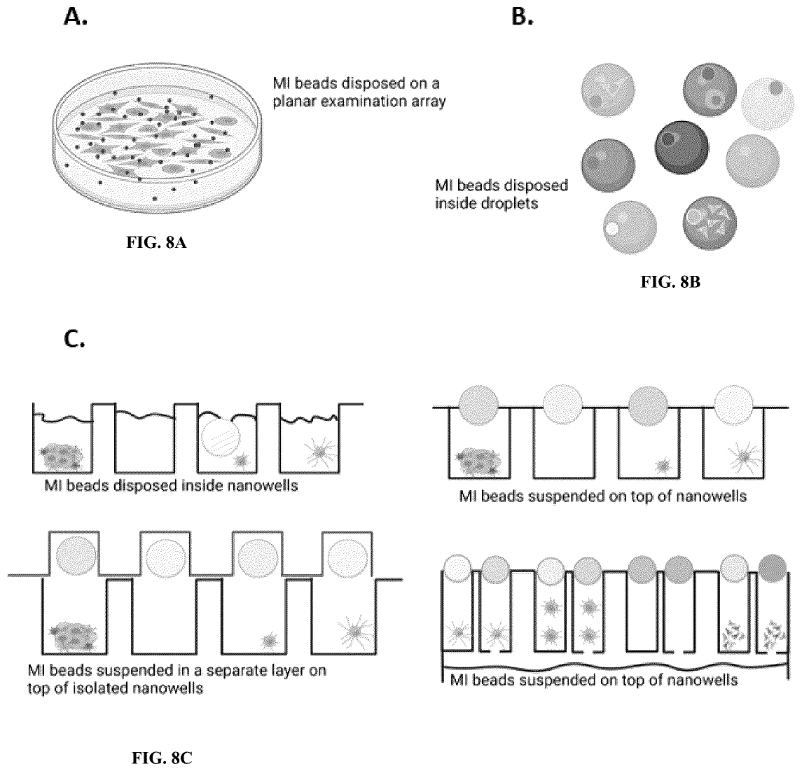

In an example, capture oligonucleotides may be covalently attached to at least one of the plurality of capture beads. Alternatively, or in addition, the plurality of oligonucleotides attached to the capture bead are cleavable. Cleaving the oligonucleotide may comprise, for example, optical cleavage, chemical cleavage, enzymatic cleavage, mechanical cleavage, acoustic cleavage, thermal cleavage, and/or pressure cleavage. For example, at least one of the plurality of oligonucleotides attached to the capture bead and at least one of the multi-index barcodes may be cleavable and cleaving may be performed at the same time as the release of nucleic acid from cells in the examination area. Further, at least one index from the multi-index barcode may be cleaved and the index may additionally be captured by a capture bead via the same mechanism as the capture of nucleic acid released from the cell in the examination area. In some examples, the plurality of oligonucleotides attached to a capture bead may be identical. In other examples, the plurality of oligonucleotides attached to a capture bead may be non-identical. Alternatively, or in addition, at least one of the plurality of oligonucleotides attached to the capture bead may not be cleavable. The capture bead may be within the examination area, on the examination area, above the examination area, adjacent examination area, and/or under the examination area (as shown in FIG. 8A-C).

An example of one of the plurality of capture beads may have more than one oligonucleotide attached and further at least a portion of the attached oligonucleotides may include an identical sequence. In addition, identifying at least one capture bead in the plurality of capture beads may comprise identifying the identical oligonucleotide sequence, wherein the identical sequence is unique to the at least one capture bead. Alternatively, or in addition, at least one of the plurality of capture beads may have more than one oligonucleotide attached and the attached oligonucleotides may include different sequences. Identifying at least one capture bead in the plurality of capture beads may, for example, comprise identifying the different oligonucleotide sequences are unique to the capture bead.

In another example, the sequences of at least one of the oligonucleotides and the plurality of nucleotides of at least one capture bead in the plurality of beads may be different. In addition, at least a portion of the sequence of the plurality of capture oligonucleotides may be different. In still other examples, at least a portion of the oligonucleotide sequence of the plurality of capture oligonucleotides may be identical.

An example of the assay performed in at least one of the plurality of examination areas may include an imaging assay, a morphology assay, a cell-painting assay, a luminescence assay, a fluorescence assay, a thermal shift assay, a nucleic acid hybridization assay, a FISH assay, a cytokine release assay, an antibody release assay, an antibody detection assay, an immunofluorescence assay, a cellular co-culture assay, a cellular growth assay, a cytotoxicity assay, a proliferation assay, a cell killing assay, a protein degradation assay, an enzyme inhibition assay, a protein-protein interaction assay, a protein-small molecule interaction assay, and/or a protein-nucleic acid interaction assay. Collecting information from at least one of the indices of the multi-index barcode and collecting information from the assays may occur concurrently. Alternatively, or in addition, collecting information from at least one of the indices of the multi-index barcode may occur before collecting information from the assays or after collecting information from the assays. Further, information collected from one of the indices of the multi-index barcode may comprise, for example, image, size, shape, pictorial, optical barcode, charge and/or sequence.

In some examples, the nucleic acid and/or index of the multi-index barcode may be released by, for example, introducing a reducing agent, a detergent, a protease enzyme, a protein denaturing agent, an apolar solvent, an alcohol, a chaotropic agent and/or a cellular lysis agent. Another example of releasing the nucleic acid and/or index of the multi-index barcode includes thermal denaturation and/or performing repeated cycles of freezing and thawing.

Capturing nucleic acid released from the cell and the released index may include, for example, hybridization induced by sequence complementarity between the nucleic acids released from the cell and the capture oligonucleotides on the capture bead, and the barcodes released from the multi-indexed barcode bead and the capture oligonucleotides on the capture bead (FIG. 5D). In addition, analyzing the capture bead may comprise sequencing the capture bead. For example, the sequencing of the capture bead may include sequencing the captured oligonucleotides. Further, sequencing the captured oligonucleotides may also comprise sequencing the multi-index barcodes and cellular nucleic acids attached to the capture oligonucleotides. Alternatively, or in addition, capturing one or more nucleic acid from a cell may comprise capture oligonucleotides having nucleic acid, wherein the nucleic acid includes DNA, RNA, mRNA, microRNA, long non-coding RNA, circular RNA, exogenous RNA, guide RNA, siRNA, and/or shRNA.

A molecular-functional profile may be generated from the information collected from the captured nucleic acid from the cell, information captured from the multi-index bead and functional data gathered from an assay performed in the examination area. For example, the molecular-functional profile may include combining morphology of the cell and a transcriptional profile. Alternatively, or in addition, the molecular-functional profiling may comprise combining an image of the cell (or an imaging assay of the cell) and a transcriptional profile.

Another example of a method for analyzing a plurality of capture beads may comprise exposing the capture beads to a plurality of cells, wherein at least one of the plurality of cells is in at least one of a plurality of examination areas; collecting the capture beads, wherein each capture bead further comprises at least one of a perturbation agent, a plurality of capture oligonucleotides, genomic content captured from the cells exposed to the bead in the examination area, a multi-index barcode, a spatial-index barcode, and/or a plurality of non-nucleotide tags, sequencing the capture bead; and generating an identity associated with at least one capture bead, wherein the generated identity includes the sequence of the genetic content captured from at least one cell and the capture oligonucleotide, and wherein sequencing the capture bead includes sequencing the capture oligonucleotide and the genomic content of the capture beads, and wherein the sequence of the genetic content includes nucleic acid captured from cells exposed to the capture beads, and the generated identity further comprises synthesis history of the perturbation agent on the bead and/or spatial location of the cells exposed to the beads.

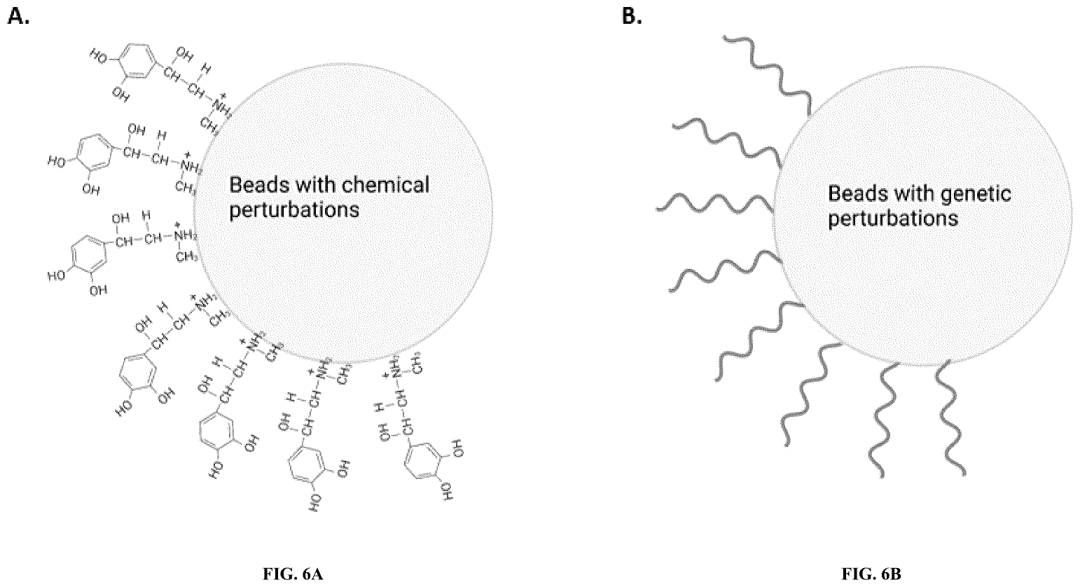

An example of beads with perturbations is shown in FIG. 6A-B. The perturbation agents may belong to a library of perturbations. The library of perturbations may comprise, for example, genetic perturbations, chemical perturbations, peptide perturbations, protein perturbations, cellular perturbations, antigen perturbations, antibody perturbations and/or solvent perturbations (FIG. 5A-D). In some examples, perturbation may be synthesized on the plurality of capture beads. Alternative, or in addition, perturbations may be pre-synthesized before attachment to the plurality of capture beads. In another example, one or more perturbations may be controllably releasable from the plurality of capture beads. For example, perturbations may be controllably released via optical release, chemical release, enzymatic release, mechanical release, acoustic release, thermal release, pressure release and/or diffusive release. In addition, the amount of releases perturbation may be measure using, for example, optical measurements, mass spectrometry, thermal, fluorescence, absorbance and/or chemical reaction.

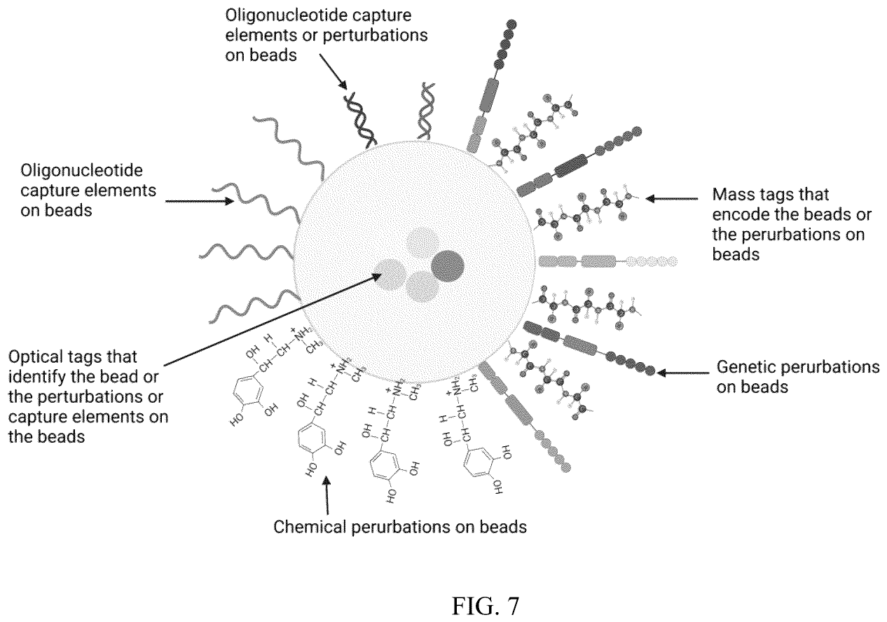

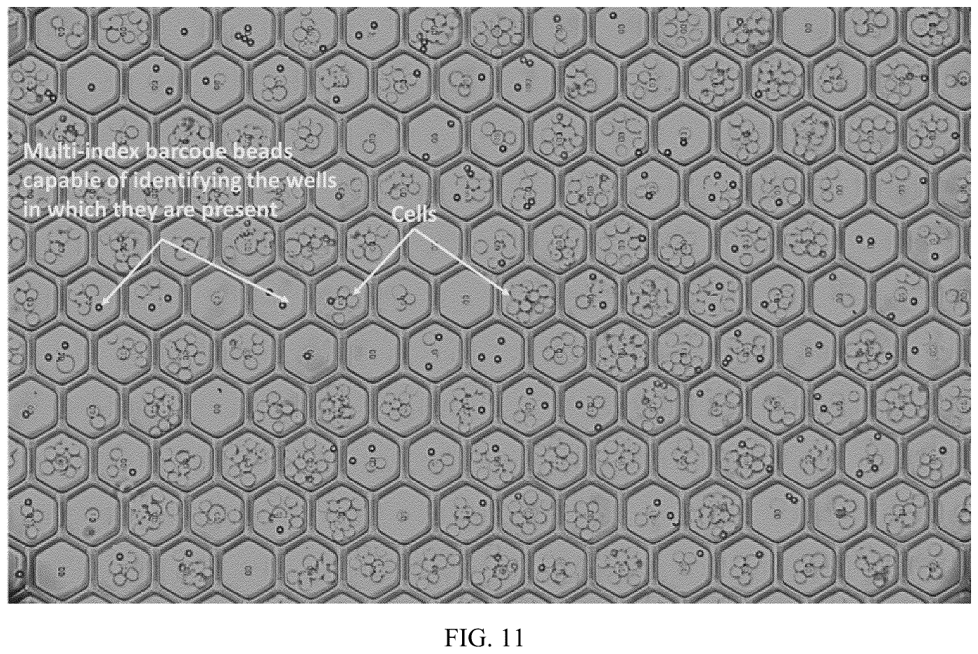

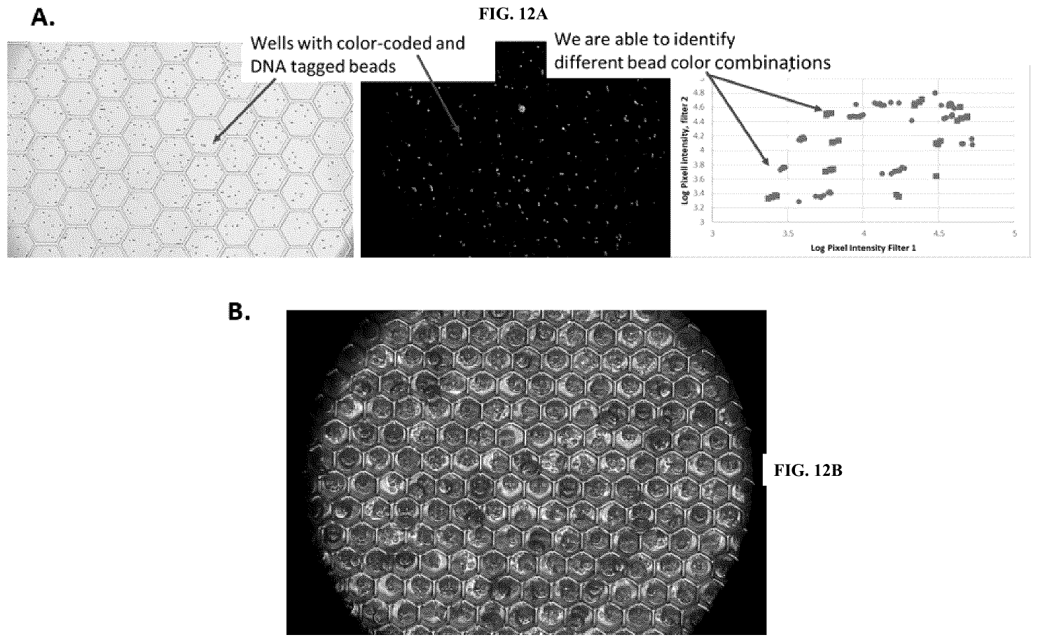

Referring to FIG. 7, a multi-index barcode bead may have perturbations and capture elements. Alternatively, or in addition, the multi-index barcode may comprise a spatial-index and the spatial-index may further include an oligonucleotide index. For example, identifying the location of the examination area may comprise contacting the examination area with at least one of the plurality of beads, wherein the bead is identifiable by a captured spatial index (FIG. 11).

Perturbations may be delivered to examination areas via perturbation beads. The perturbation beads may be disposed proximal to the examination areas such that cells in the examination areas are capable of being contacted by the perturbations (shown in FIGS. 8A-D). Further, FIG. 8A illustrates perturbation beads that may be disposed on the same surface as the cells in the examination areas. In other examples, and as illustrated in FIG. 8C, the perturbation beads may be disposed in a layer above the cells in the examination area. The perturbation beads may also be disposed in an area below the cells in the examination area. In yet other examples, as seen in FIG. 8B, the perturbation beads may be disposed inside droplets.

An example of the perturbations synthesized directly on the perturbation beads may be accomplished by means such as split-pool combinatorial chemistry. Various methods to perform split-pool combinatorial synthesis are well known to people familiar in the art, and any one of those means may be used to generate perturbations on the beads. Another example may be that the perturbations are attached to the perturbation bead or synthesized on the perturbation beads via cleavable linkers, such that the perturbations may be released from the beads by cleaving the linker. For example, the cleavable linker may comprise a photocleavable linker such as a nitro-benzyl containing linker, or another photocleavable linker. Further, the amount of perturbations released from the perturbation bead may be controlled by controlling the amount of photo-stimulation applied to the beads. In other examples, the cleavable linker may be a thermal cleavable linker, an enzyme cleavable linker, a chemical cleavable linker, other suitable cleavable linkers and/or a combination thereof.

The perturbation beads may comprise bead substrates such as monosized TentaGel® M NH2beads (10, 20, 30, etc., micrometers in diameter)—, standard TentaGel® amino resins (90, 130, etc. micrometers in diameter), TentaGel Macrobeads® (280-320 micrometers in diameter) (all of the above from Rapp Polymere, 72072 Tubingen, Germany). These have a polystyrene core derivatized with polyethylene glycol (Paulick et al (2006) J. Comb. Chem. 8:417-426). TentaGel® resins are grafted copolymers consisting of a low crosslinked polystyrene matrix on which polyethylene glycol (PEG) is grafted. Thus, the present disclosure provides beads or resins that are modified to include perturbations, where the unmodified beads take the form of grafted copolymers consisting of a low crosslinked polystyrene matrix on which polyethylene glycol (PEG) is grafted.

In some examples, perturbations may be encapsulated within pores or chambers or tunnels within the beads, without covalent attachment to the beads. Perturbations may be diffused into or forced within such pores of the beads by various means. For example, where the perturbations are loaded onto beads without covalent attachment, the perturbations may be unloaded from the bead by way of diffusion. Alternatively, or in addition, temperature, pressure, solvents, pH, salts, buffer or detergent or combinations of such conditions may be used to unload perturbations out of such beads. Further, the physical integrity of the beads, for instance by uncrosslinking polymerized beads, may be used to release perturbations contained within such beads.

Various method for synthesizing and screening on-bead perturbation libraries are well known to practitioners of the art. One method for synthesizing and screening perturbation libraries in droplets is elaborated in (MacConnell, et al. ACS Comb Sci. 2015 Sep. 14; 17(9): 518-534) paper. One method for cell bases screening of bead perturbations is described in (Cho, et al. ACS Comb. Sci., 2013, DOI: 10.1021/co4000584) paper. Methods for generating and screening perturbation beads in microwell and picowell arrays are elaborated in Plexium patent portfolio. Methods for identifying combinatorially generated perturbation libraries by mass spectroscopy are elaborated in Application PCT/EP1997/002215 patent application.

In addition to beads, vesicles or droplets may also be used as vehicles for delivering perturbations proximal to examination area for some embodiments of the present disclosure. Lipids, diblock-copolymers, tri-block copolymers or other membrane forming materials may be used to form an internal volume into which perturbations may be loaded. Perturbations may be released from these encapsulated volumes by addition of detergent, mechanical agitation, temperature, salt, pH or other means. Water-in-oil droplet emulsions or oil-in-water droplet emulsions are yet other means to passively encapsulate perturbations that may be delivered to assay volumes.

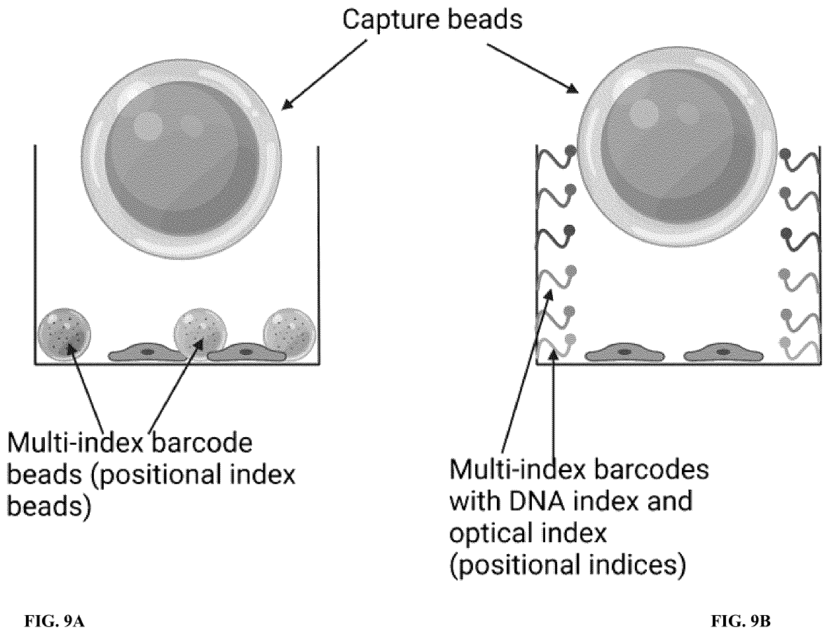

Referring to FIG. 9, cells may be grown, or allowed to multiply in examination areas, such that should an examination area be disposed of a single cell, said examination area would be disposed of clonal copies of the original cell after the process of cell multiplication. Additionally, examination areas may comprise clonal copies of a single cell. An example of an examination area may comprise heterogenous populations of cells. Further, in some examples, the clonal copies of cells are in fluidic contact with each other but fluidically isolated from other cells (see FIG. 8A-C). Further, clonal copies of cells may be in fluidic contact with clonal copies of other cells and/or the clonal copies of a cell may be in fluidic contact and fluidically isolated from some of the other clonal copies of the same cell.

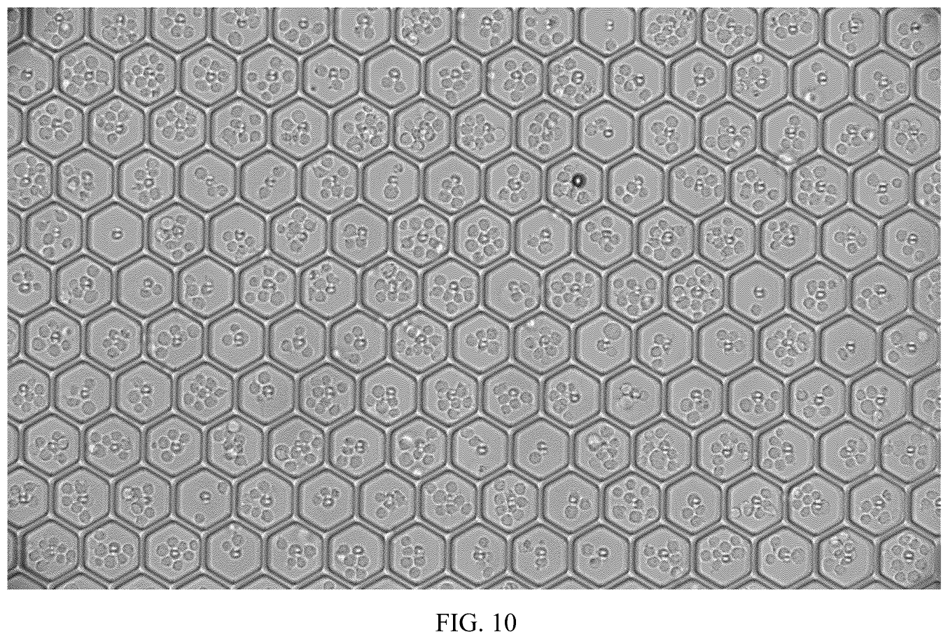

The cells in the examination area may be imaged optically. For example, the cells may be imaged prior to their multiplication or after they are allowed to multiply. Alternatively, or in addition, the cells may be imaged before, after or even during the process of cell division (or multiplication, or cell culture). Further, the cells may be induced to multiply by the addition of growth promoting factors, reagents or additives. The cells may also be induced to multiply by, for example, the addition of other cells that induce proliferation of the cells in the examination areas. Imaging of the cells may allow for counting of cells in the examination areas. In addition, imaging of the cells may allow for estimating the rate of proliferation of cells in the examination areas. FIG. 10 shows an example image of nanowells populated with cells.

Another example of a method to analyze cells of the examination area may be probing by probing agents external to the cells. For example, the cells may be probed by one or more of a collection of antibodies capable of binding to intact components or denatured components of cells (shown in FIG. 5). In some examples, the cells may be probed by external agents, and the cells disposed in examination areas may be intact, live cells. Alternatively, or in addition, the cells are probed by external agents may be fixed or denatured cells. The external agents may be, for example, chemical agents. Alternatively, or in addition, the external agents may be intended to generate light, fluorescence, scattering or other optically resolvable signals from the cell. Further, the external agents may be proteins, enzymes or other biologically active materials. In yet other examples, the external agents may be nucleic acids, oligonucleotides, nucleotides or other agents capable of binding to or probing components of the cell. The external probing agents may also be combination probing agents comprising one or more of protein, chemical, or nucleic acid components, where some of the components of the probe may bind to cellular material whereas some other components of the external probe may be used to read-out the binding of the probing agent to the cells. In some examples, the cells may be permeabilized to allow entry of external agents into the cell. In addition, cells may be exposed to probe molecules including nucleic acid handles. Probe molecules may further comprise, for example, antibodies, aptamers, small molecules, bifunctional molecules, quantum dots, beads, and/or unnatural nucleotides deigned to probe contents of the cells. In an example, capturing at least one capture oligonucleotide may comprise the capture oligonucleotide attaching to at least one nucleic acid handle and/or probe molecule.

The external probing agents may be used to probe, for example, the presence of level of one or more proteins in cells. In addition, the external probing agents may be used to probe the presence or levels of one of more genomic components of the cell. Alternatively, or in addition, the external probing agents may be used to probe one or more metabolic components of cell, the physical location of various components of the cell, such as for example, the organelle, nucleic acid and mitochondria and/or to examine the morphology of the cells.

In an example, the cells may be examined by endogenously expressed reporters. The endogenous reporters may be optical reporters. The optical reporters may be, for example, fluorescent proteins and/or portions of luminescent proteins. Alternatively, or in addition, the cells may be examined for the presence and amounts of a number of endogenous reporters. For example, the cells may be examined for secreted molecules, secreted proteins, and/or secreted metabolites. Further, the endogenous reporters may report the presence or absence of specific interactions within the cells.

Different cells in different examination areas may be probed for, or report, different features being examined. For example, different cells may be probed for presence or amounts of different components of the cell. In addition, different cells may be probed for the reporting of different interactions within the cell. The reporter cells may also report protein-protein interactions.

Referring again to FIG. 8A-D, cells in different examination areas may be exposed to different perturbations. For example, the cells in different examination areas may be exposed to different perturbations from a library of perturbations. In some examples, the library of perturbations is known; whereas, in some other examples, the identity of the perturbations in the library of perturbations may not be known at the time of exposing the cells to the perturbations.

For example, live cells in the examination area may be perturbed by external perturbations. The perturbations may be, for example, oligonucleotide perturbations and/or small-molecule chemical perturbations. Cells may be examined after being exposed to perturbations. For example, where the cells are perturbed by external perturbations, the cells may be examined by external probes. Alternatively, or in addition, where the cells are perturbed by external perturbations, the cells may be examined for endogenous features or reporter signals. Some examples of cellular assays in microwell and nanowell arrays are reported (Torres, et al., Anal. Chem. 2014, 86, 11562-11569).

An example of this disclosure may include the functional state of cells or the response of cells to perturbations delivered by perturbation beads that may be captured for further analysis. For example, the state of the cells or the response of the cells to perturbations may be captured on capture beads disposed proximal to the cells (FIGS. 8A and 8C and FIG. 9A). The capture beads may be the same as the perturbation beads. In addition, the measured cell state or change in cell state may comprise measuring levels of proteins and comparing to a reference cell. Further, the changes from the reference cell may include, for example, changes in levels of proteins, changes in morphology, changes in cellular localization of proteins and genetic materials in the cell, changes in the size of cells, changes in RNA levels of cells, changes in mRNA levels of cells, changes in genetic state and composition of cells, changes in secreted materials from the cells or changes in cell-cell interactions. As previously described, the cellular state or cellular responses to perturbation may be captured by addition of probes.

As one example, the genetic content of cells in the examination area can be captured for further analysis. For example, the genetic content may comprise one or more of DNA, mRNA, RNA, microRNA, non-coding RNA, circular RNA, long-non-coding RNA or other nucleic acids from the cells. The genetic content of the cells may be captured in response to perturbations delivered by perturbations beads proximal to the cells (see generally, FIGS. 8A-C and 9A-B). The captured genetic material may represent, for example, the functional state of the cell. The genetic content of the cells may, for example, be captured on capture beads, wherein the capture beads are suitably functionalized to capture the genetic content of cells. In some examples, each capture bead may have a plurality of oligonucleotides attached and the oligonucleotides attached to each capture bead may uniquely identify the bead from a population of other capture beads. Alternatively, or in addition, each capture oligonucleotide on each capture bead may also comprise a segment that uniquely identifies that particular oligonucleotide from the other oligonucleotides on the same bead. In addition, a plurality of capture oligonucleotides on each bead may comprise a segment that can hybridize to complementary nucleic acid sequences of cells. For example, the complementary sequence may be a poly-T sequence capable of hybridizing to a 3′ poly-A sequence of mRNA molecules inside a cell. The cells may be lysed to capture their genetic content onto capture beads. Cell lysis may be done by one or more ways familiar to people trained in the art. Such methods may comprise addition of detergents, specially formulated lysis buffers, thermal rupture, freeze-thaw thermal cycling etc.

In another example, capture beads may capture non-genomic analytes from cells. Further, the capture beads may capture proteins from the cells. The proteins may be, for example, secreted proteins such as cytokines. In still other examples, the capture beads may capture metabolites secreted from the cells. For example, the capture beads may capture content from lysed cells after a lysis step, and such content may comprise proteins, metabolites, lipids or other analytes. Alternatively, or in addition, the capture beads may be different from perturbation beads. In addition, the capture beads and perturbation beads may be disposed in proximity to cellular examination areas (FIG. 8A). Material captured on capture beads may be analyzed, for example, to understand the properties of cells. For example, analysis methods may comprise protein analysis, metabolite analysis or other analysis of cellular content, wherein further, the analysis technology may comprise mass spectroscopy, fluorescence labeling of capture content, antibody staining, ELISA, or other detection methods. Alternatively, or in addition, multiple capture beads may be present in proximity to an examination area such that different capture beads may capture different analytes.

In some examples, the capture beads may be the same as the perturbation beads that deliver perturbations to cells in the examination areas. Creating perturbations on capture beads may comprise encoded capture beads. Methods to create perturbations on capture beads are well known to people familiar with the art. DNA-encoded combinatorial libraries (DELs) have gained prominence as a scalable method to create vast chemical libraries. Solid-phase versions of DELs, for example DNA-encoded one-bead one-compound libraries are an especially powerful version of DELs as they allow cell-based experiments to be performed when the compounds are linked to beads through a cleavable linker. Publication such as MacConnell et, al. ACS Comb. Sci. 2015, 17, 9, 518-534, DOI: 10.1021/acscombsci.5b00106 and US patent application US20190093103A1 (incorporated herein in its entirety) envision multiple copies of one encoding nucleotide identifying the synthetic history of the compounds on the beads (represented as ‘substantially identical’ DNA barcodes in US20190093103A1, where ‘substantially’ is elaborated in the specification para [0040] to be at least 70% or more identical, and further in [0353] beads with less than 50% identical DNA barcodes are suggested to be excluded as unsuitable). There are serious limitation to using substantially identical DNA barcodes to represent the synthetic history of compounds synthesized on beads. For example, as clearly established in Sauter, et, al, Bioorganic & Medicinal Chemistry, Volume 52, 15 Dec. 2021, 116508 DOI: 10.1016/j.bmc.2021.116508, many of the common synthetic reactions are damaging to the DNA (for example FIG. 1 of the cited publication suggests only about 30% of encoding DNA survives one cycle of a Suzuki reaction. For a 3 cycles involving DNA barcodes, only 2.7% of the DNA are expected to survive at the end). More importantly, the publication explains that the damage to the DNA is caused by different mutations induced by the chemical reagents, and that different nucleobases are susceptible to mutation under different reaction conditions. This mutation pattern may change further based on the chemical building block that is involved in the synthesis reaction, thereby creating a sequence dependent DNA mutational pattern for DNA-encoded chemical libraries.

In some example, and as shown in FIG. 12A, optical barcodes on multi-index beads may be used to uniquely identifying individual wells of a nanowell array. The optical barcodes of the multi-index barcode beads may be deposited into the nanowell examination areas and decoded. For example, each bead may be one of 20 possible beads, where the 20 bead barcodes are identifiable by the ratio of two colors. FIG. 12B provides an illustration of capture beads disposed into examination areas that contain cells and multi-index barcode beads. In some embodiments, optical tags may be employed to identify the oligonucleotide tags that are released from the multi-index beads and captured by capture beads.

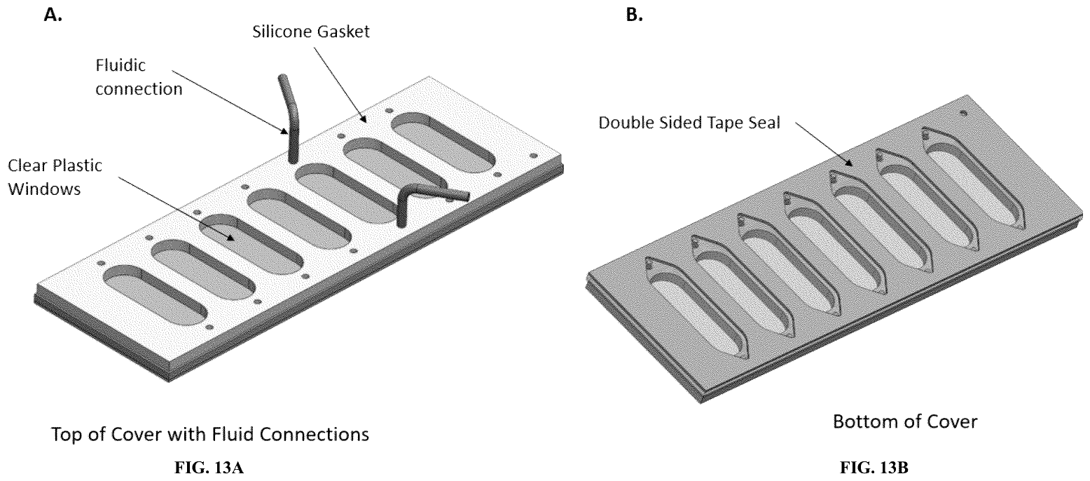

Referring now to FIG. 13A, in some examples, the top surface of a lid is used to create a reversibly enclosable examination chip. Further, the top surface may contain a pliant silicone gasket that will contact the top portion of a clamp that will be screwed to the bottom portion of the clamp. Alternatively, or in addition, the pliant material may be placed atop a clear optical window to allow light passage and optical interrogation of the chip enclosed by the clamp. In some examples, the lid may also include holes that allow fluidic connection to the examination areas enclosed by the reversibly enclosable clamp. FIG. 13B shows the bottom surface of a lid that may be used to create a reversibly enclosable examination chip. In some embodiments, the bottom surface of this lid may press against an examination chip, which may in turn press against the bottom portion of a clamp.

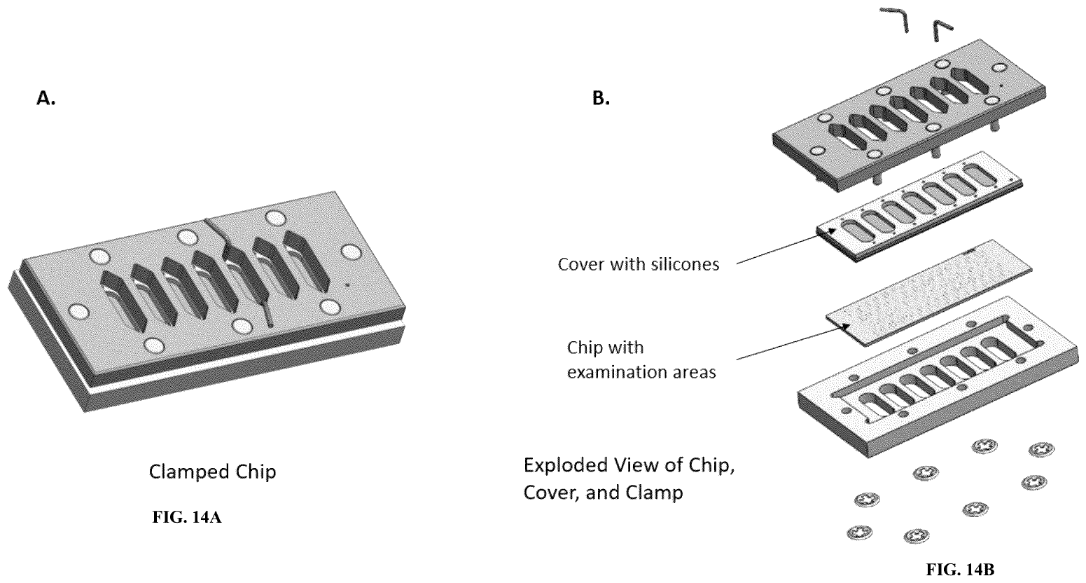

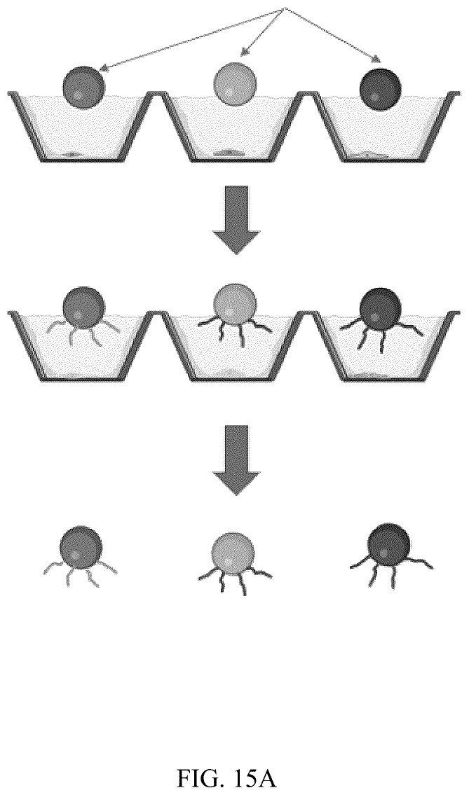

Similarly, FIG. 14A illustrates the closed form of the clamp containing an examination chip sandwiched between a lid with optical windows and the top and bottom portions of the clamp. As seen in FIG. 14B, the clamping architecture of the examination chip may sit under a lid which may then be clamped together by the top and bottom portion of the clamp.

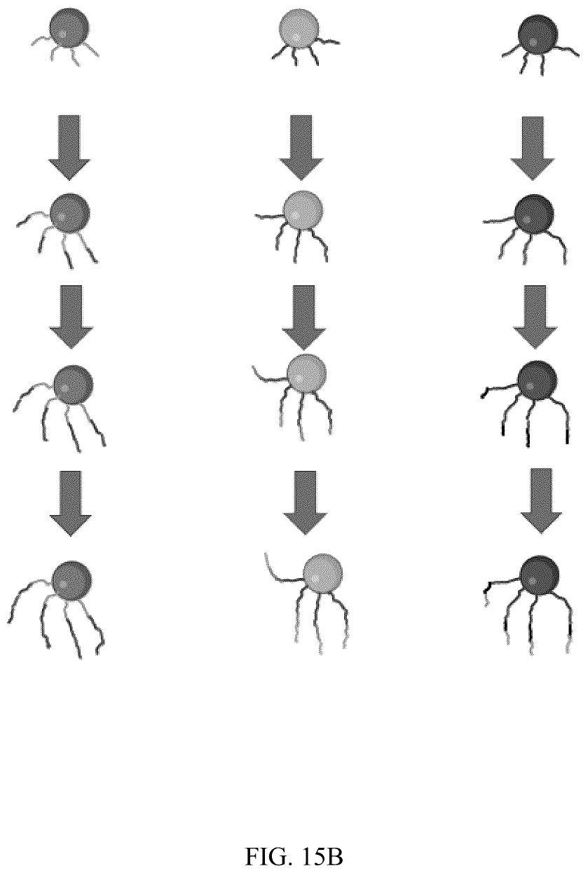

In some embodiments, non-distinguishable beads may be used for single-cell barcoding. Accordingly, FIG. 15A shows a non-distinguishable bead placed in an examination area with a cell of interest. When the cell of interest is lysed, nucleic acid content may be released from the cell and captured by the non-distinguishable bead. Beads may then be extracted from the examination area. In addition, FIG. 15B shows the process of synthesizing a bead-unique barcode using split-pool oligonucleotide extension.

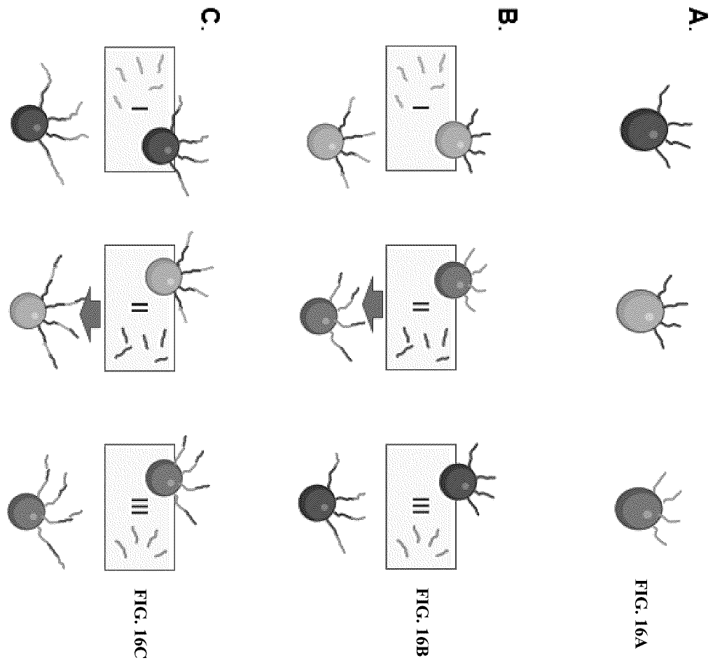

Moreover, ledger synthesis may be included to correlate optically barcoded beads to a spatial position as seen in FIG. 16A. FIG. 16B shows the progress of the ledger synthesis after a first split. As seen in FIG. 16B, the Orange bead is appended with barcode fragment I, the Teal bead is appended with barcode fragment II, and the Blue bead is appended by barcode fragment III. In some examples, the ledger synthesis may include one or more additional splits. For example, FIG. 16C shows ledger synthesis after optical imaging of a second split: the Orange bead is appended with barcode fragment II, the Teal bead is appended with barcode fragment III, and the Blue bead is being appended by barcode fragment I.

As contemplated by other approaches, using ‘substantially identical’ or identical DNA encoding tags, therefore, cause severe mutational burden on the encoding tags, severely limiting the number and fidelity of DNA barcodes that survive multiple cycles of synthesis in the presence of a diverse set of chemical building blocks and conjugation conditions. In preferred examples of this disclosure, any encoding nucleotide tags used on the beads of this disclosure are designed to be substantially diverse as opposed to ‘substantially identical’. Substantially diverse oligonucleotide tags may comprise oligonucleotides of substantially differing sequence composition. In some examples, at least 60% of the nucleotide barcodes on a bead that encode the perturbations on that bead are different from each other. In some examples less than 30% of the perturbation encoding oligonucleotide tags on a bead are reasonably identical, reasonably signifying 90% of more sequence similarity. The advantages of using a diverse set of nucleotide tags to encode the perturbation on a bead is that while one sequence of encoding tag may get mutated in a synthetic step, a different sequence of DNA may be less susceptible to damage, thereby more correctly identifying the perturbation on the bead.

In some examples the diversity of encoding DNA tags are generated by utilizing a diversity of barcodes that uniquely identify the chemical building blocks of the combinatorial synthesis reaction. For instance, using 5 different and substantially dissimilar DNA barcodes to represent each chemical building block would result in 5×5×5=125 different DNA barcodes that identify a perturbation on a bead that has undergone 3 cycles of combinatorial synthesis. In this specific example, the encoding DNA tags on the bead are less than 1% identical to each other, providing at least 99% higher fidelity to DNA damage on a per-tag basis. This redundancy and corresponding fidelity can substantially expand the scope of chemical reactions and building blocks that can be subject to DNA encoded combinatorial libraries on beads. In one example the capture beads contain oligonucleotide encoded chemical perturbations, wherein the plurality of perturbation-encoding oligonucleotides on a given bead are substantially different from one another to ensure fidelity and redundancy of encoding, wherein further the oligonucleotides that encode the chemical perturbation are suitably functionalized to also serve as the capture oligonucleotides of the capture beads (for instance, by possessing a poly-T terminus that can capture 3′ poly-A tails of mRNA or by possessing sequence specific termini that capture select complementary nucleic acids from an experimental milieu).

In some examples of this disclosure the perturbations on the beads may be recorded in barcodes by other means different from oligonucleotide encoding. For instance, the perturbations on the beads of this disclosure may be recorded via optical barcodes or by optical encoding. Methods for generating optically barcoded beads or optically encoded beads are well known to practitioners of the art, for instance as illustrated in US20070161043A1 and WO2021042011A1, incorporated herein in their entirety by reference. In such examples it is still advantageous to display capture oligonucleotides on these perturbation-bearing beads so that genomic and molecular responses of cells to perturbations may be captured on the same bead that delivered the perturbation. In other examples, it is advantageous for the multiple indices on a multi-index bead to be related to each other such that the information contained in one index could identify the information contained in the other index. Generalized methods to create and track the multiple encoding tags or indices on a bead can be accomplished by a method called ledger-synthesis.

In the process of ledger-synthesis any modification made to a bead is appended to a ledger of bead-information, where a bead is tracked on the ledger by means of one of more measurable information from one or more indices or barcodes on the bead. Examples of processing beads may undergo include chemical synthesis, oligonucleotide barcode synthesis, optical encoding, mass encoding, nucleic acid synthesis, washing, imaging, PCR, capturing genomic content or molecular signatures from cells in examination areas, micromanipulation using a micromanipulator, and the like.

In one example, oligonucleotide barcodes may be added to an optically encoded library of beads using ledger-synthesis (said optically encoded beads may or may not contain perturbations, or the perturbations may be added to the optically encoded beads also by means of ledger synthesis). In one example, a population of M number of optically encoded beads is distributed into N compartments (each compartment receiving roughly M/N number of beads, or another ratio or a random number of beads), where each compartment contains a known oligonucleotide tag capable of being attached to the subset of beads present in that compartment. By imaging the set of beads contained in each of the compartments and knowing the sequence of the oligonucleotides present in each compartment, the bead-information ledger is updated to indicate the oligonucleotide barcodes now attached to each bead. Equivalently, the optical barcodes of each bead are now appended on the ledger with the oligonucleotide barcodes attached to the beads. The process of attaching oligonucleotide barcodes to beads is well known to people familiar with the art, where the attachment may be affected through a cleavable linker or a non-cleavable linker. To create longer or more complex oligonucleotides, a split-and-pool strategy may be employed as part of ledger-synthesis. In one example, after the first step of oligonucleotide attachment, all the beads in the various compartment may be pooled together, mixed thoroughly, and a new random set of beads may be distributed into each of the N oligonucleotide containing compartments. The new set of beads in each compartment are imaged, and the corresponding bead information is appended on the ledger with the new oligonucleotide tag being attached to the bead. In this example, the ledger would now contain two oligonucleotide sequences along with the optical barcode from each of the beads. The oligonucleotide barcodes from the two splits may be attached to each other (for instance via ligation or polymerase extension, methods familiar to people in the art) or exist separately on the beads. One method for oligonucleotide extension on beads using the split-and-pool approach is described in Delley et, at., Sci. Rep. 2021; 11: 10857, DOI: 10.1038/s41598-021-90255-x. This cycle of split-and-pool synthesis may be repeated multiple times and the bead ledger appended each time with the new information added to the bead. The advantage of ledger synthesis is the ability to use one measurable index on the bead to identify other indices on the same bead. In the example above, the optical barcode on the beads can indicate the sequence of oligonucleotide barcodes added to the bead by way of the ledger. In some examples, there may be a plurality of oligonucleotides in each compartment, say Q number of different oligonucleotide tags in each compartment such that after 3 cycles of split-pool synthesis, there would be QxQxQ number of distinct oligonucleotide tags on each bead. The ledger of bead information is able to relate each, or any, of the QxQxQ nucleotides to the optical barcodes on the beads. Such redundancy is crucial to maintain fidelity of oligonucleotide barcodes subject to various types of bead processing or manipulation.

In another example, chemical synthesis may be performed on optically encoded beads to generate optically encoded chemical perturbation libraries. In this ledger-synthesis process, a set of N optically encoded beads are first split into M compartments, each compartment containing one chemical building block capable of being attached to the bead through a cleavable linker. Well know methods for on-bead synthesis may be employed to attach this first building block to the beads, for example using bead functionalization and building block conjugation methods described in US20190093103A1, incorporated herein in their entirety by reference. By imaging the optical barcodes on the beads in each of the M compartments, the ledger of bead-information is appended to include the chemical building block attached to each of the detected optically encoded beads. After suitable wash and filter steps to remove unreacted building blocks that may be stuck on the beads, all the beads from the M compartments may be pooled together, mixed thoroughly, and split once again into the M compartments (or compartments of another number) optionally containing a different set of chemical building blocks capable of reacting to the first building blocks attached to the beads in the previous reaction step. By imaging the optical barcodes on the beads in each compartment, the bead-information ledger is again appended to now include the second building block attached to each of the optically encoded beads. Performing this split-and-pool steps multiple times allows the optical barcodes on a bead to identify the synthetic history of compounds present on the bead. This ledger-synthesis example illustrates how the library of on-bead chemical perturbations can be identified using the optical indices present on the beads. Notably, the compounds are not optically encoded (each chemical building block does not correspond to a single optical tag), rather the bead-information ledger tracks synthetic history of each bead using its optical barcodes.

In some examples, a single index is used to track multiple types of processing steps on the beads. In one example, optical barcodes on beads may be used to track the synthetic history of chemical synthesis on the beads as well as the oligonucleotide barcodes attached to the beads in a separate processing step. In some examples the multi-indexed barcode beads of this disclosure are generated by the process of ledger-synthesis. In some examples the capture beads of this disclosure are generated by the process of ledger-synthesis. In some examples, the optical barcodes on a multi-index bead identifies the position of the bead in an examination array or examination chip, wherein further the oligonucleotide barcode on the same bead is released to be captured by a barcoded capture bead proximal to the examination area along with nucleic acids from cells in the examination area, thereby identifying the position of the cells in the examination array using nucleotide sequences alone, and enabling a molecular-functional profile of cells.

In one example, ledger-synthesis is used to generate oligonucleotide barcodes on optically encoded capture beads already containing nucleic acids captured from cells. In this example, optically barcoded beads comprising non-barcoded oligonucleotide capture elements are used as capture beads to capture nucleic acids from cells. (Such non-barcoded capture elements may comprise poly-T sequences or other sequences complementary to cellular nucleic acids of interest, such that the various capture beads are distinguishable only by their optical barcodes and not the nucleic acid capture elements, which are identical on all beads). In this example, nucleic acids from cells are captured on the non-barcoded nucleotides on the beads. Said captured cellular nucleic acids are subsequently functionalized with a universal sequence at their terminal end (3′ end) from which bead-specific oligonucleotide barcodes may be synthesized via ledger-synthesis tracked by the optical barcodes on the beads. Universal functionalization of bead anchored nucleic acids may be performed by many ways known to people familiar in the art. For instance, by ligation of a universal oligonucleotide tag or in the case of case of captured RNA, using a process called template-switching, conveniently available as kits from various vendors.

The examination areas of this disclosure may be enclosed within a chamber comprising a top for a certain time (for example when performing an assay), whereas at other times the examination area may be open to the atmosphere (for example when retrieving cells or beads from the chips for further analysis). FIGS. 13A-B and 14A-B provide an illustration of how such a reversibly enclosable chamber may be constructed. The design and fabrication of such mechanical features will be well known to people familiar in the art of mechanical engineering and design. As illustrated in FIG. 14B, the examination chip, in this illustration comprising an array of nanowell examination areas, is compressed between the top portion and the bottom portion of a clamp with a silicone-padded lid with optical windows providing a measure of control over the dimension of the chamber the examination areas are enclosed within. The lid also provides fluidic access to the examination areas such that fluids containing cells, beads, reagents or other analytes required for molecular-functional assays may be exchanged within the fluidic chamber that is created.

In some examples, the lid of FIGS. 13A-B and 14A-B may be planar lid with no gaskets on its top or bottom. In some examples the examination chip may be fabricated to comprise a raised edge, with the array of examination areas recessed by a certain distance from the raised edge of the examination chip. Such examination chips containing features of different heights in the same (or, optionally, different) material may be readily created by various fabrication processes such as multi-step photolithography, multi-step photolithography followed by hot-embossing, micromachining, or any mechanical fabrication processes that allow creating features of different heights or depths. There is a substantial advantage to creating examination chips with intrinsically raised edges or raised features as it eliminates an alignment step needed to affix an intermediate layer to space the top surface of the examination areas from the lid used to create a fluidic chamber above the examination areas. The raised edges of the examination chip also allow fluidic connection between the different examination areas as contemplated in the various examples of this disclosure.

In some examples, cells and beads of this disclosure may be introduce into the examination areas by way of a fluidic connection to the examination areas in the enclosed form of the examination chip. In some examples the cells and beads of this disclosure may be delivered to the examination area in the open form of the examination chip, wherein a lid or clamp is absent above the top surface of the examination chip. The beads and/or cells loaded onto the examination chips may be allowed to deposit into the examination areas passively by gravity or actively, for instance, by spinning the examination chip rapidly in a centrifuge.

In one example, the cells or beads contained in one examination area may be retrieved by utilizing a micromanipulator that can physically contact an examination area and retrieve contents of interest. Such micromanipulators are commercially available, for instance, the CellSelector from ALS Jena GmbH. Instructions and protocols for retrieving cells and beads from nanowell arrays are available through ALS Jena as well as through various publications that utilize this instrument. In such examples, the open form of the examination chip is utilized even if an enclosed form was utilized to load the cells and beads into the examination chip.

In some examples, the retrieved cells and/or beads from an examination area may be transferred to a macro-chamber such as a PCR tube or into one or more wells of a 96-well plate as facilitated by the micromanipulators. Further experimentation may be performed on the cells and/or beads transferred to the micro-chambers from the examination areas. In some examples, the cells retrieved from an examination area may be subject to clonal amplification and expansion to create numerous copies of the retrieved cell or cells. In some examples, the cells retrieved from the examination areas may be subject to genomic analysis to study endogenous nucleic acid content from the cell or exogenous nucleic content added before or after the cells are retrieved from the examination area (such exogenous nucleic acid comprising tags or probes introduced to the cells as illustrated in various examples of this disclosure). In some examples the genomics analysis may comprise performing PCR amplification on the nucleic acids of interest. In some examples the genomic analysis may comprise sequencing the cells retrieved from the examination areas.

In some examples, beads retrieved from one or more of the examination areas may be subject to further analysis. In one example, PCR amplification may be performed on a bead retrieved from an examination area to amplify one or more oligonucleotides found on the bead. In some examples the amplified oligonucleotides may be subject to sequencing analysis to identify the sequence of oligonucleotides contained on the retrieved beads. In one example, the retrieved beads may be subject to release conditions to release any analytes from the beads. Such release conditions may comprise photo-release, acid release, base release, physical release by vigorous shaking or crushing, release under reducing conditions, release under oxidizing conditions, thermal release, acoustic release or in a non-limiting fashion any release method capable of releasing material from the beads for further analysis. In some examples the material released from the beads retrieved from examination areas are subject to analysis by mass spectroscopy. In one example mass spectroscopy is utilized to ascertain a mass tag attached to the beads. In another examples, mass spectroscopy is utilized to ascertain the mass of any perturbagens that may be attached to the beads. One example of mass determination from perturbation containing beads is described in Avital-Shmilovici et.al, ACS Cent. Sci. 2022, 8, 1, 86-101, DOI: 10.1021/acscentsci.1c01041.

In some examples, multiple cells and multiple beads from multiple examination areas are retrieved together or individually for further analysis. In some examples entire content of examination chips may be retrieved for further analysis. In one example, the examination chip is inverted, and the content allowed to fall out of the various examination areas by gravity. In some examples mechanical force may be used to dislodge content from nanowells or microwells or other configurations of the examination areas. In some examples the enclosed form of the examination chips may be utilized to extract and retrieve content from all the fluidically accessible examination areas. In some examples magnetic beads may be used as the multi-index barcode beads or capture beads, wherein magnetic force may be used to both load and dislodge the beads from the wells.

FIGS. 15A and 15B provide an example of single-cell barcoding using non-distinguishable beads. According to one aspect, a non-distinguishable bead may not contain any nucleotide barcodes that may distinguish them from another non-distinguishable bead. The non-distinguishable bead may be placed in an examination area with at least one cell. The cell may be lysed and genomic and/or nucleic acid content may be captured on the non-distinguishable bead. For example, the genomic and/or nucleic acid content may be capture by the non-distinguishable bead via degenerate or indistinguishable capture sequences (for example, poly-T and/or unique molecular identifier with poly-T). The non-distinguishable bead with the nucleic acid content may be extracted from an examination area or well. Bead-unique barcodes may be introduced at the end of the captured genomic content by, for example, split-pool oligonucleotide extension. Split-pool oligonucleotide extension may include, for example, ligation or polymerase extensions. The genomic or nucleic acid content on each bead is uniquely encoded without the non-distinguishable beads themselves needing to have unique barcodes. Unique molecular identifiers (UMIs) may be introduced as part of the synthesized barcode.

Referring now to FIG. 16A, an example of capturing positional information from single-cells using optically barcoded beads is provided. The optically barcoded beads are similar to the non-distinguishable beads in that they do not have distinguishable nucleotide barcodes. Instead, the beads may include at least one optically distinguishable barcode. The optical barcode may allow the optically barcoded bead to be correlated with a certain spatial position. In some examples, spatial position may be an examination area or a well. For example, ledger synthesis may track the optical barcode on a ledge. Ledger synthesis may, for example, include split-pool synthesis (FIG. 16A-C). In one example, ledger synthesis comprises a first entry and may follow optical imaging of the first split. Referring to FIG. 16B, the Orange bead may be appended with barcode fragment I; the Teal bead may be appended with barcode fragment II; and the Blue bead may be appended by barcode fragment III. FIG. 16C shows a second ledger entry which may occur after optical imaging of a second split. In FIG. 16C the Orange bead may appended with barcode fragment II; the Teal bead may be appended with barcode fragment II; and the Blue bead may be appended by barcode fragment I. At the end of a few cycles of split-pool synthesis, each bead may have a unique DNA barcode appended to it. After synthesis of the unique DNA barcode on the optically barcoded bead, the DNA may be sequenced. Further, the ledger may correlate the optical barcode of the bead with the unique DNA barcode thereby associating both the optical barcode and the unique barcode with a specific spatial location.

To clarify the use of and to hereby provide notice to the public, the phrases “at least one of <A>, <B>, . . . and <N>” or “at least one of <A>, <B>, . . . or <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

Those skilled in the art will recognize, or be able to ascertain, using no more than routine experimentation, numerous equivalents to the specific procedures described herein. Such equivalents are considered to be within the scope of the inventions. Various substitutions, alterations and modifications may be made to the invention without departing from the spirit and scope of the invention. Other aspects, advantages, and modifications are within the scope of the invention.

Furthermore, the advantages described above are not necessarily the only advantages of the invention, and it is not necessarily expected that all of the described advantages will be achieved with every embodiment of the invention.

The contents of all references, issued patents, and published patent applications cited through this application are hereby incorporated by reference. The appropriate component, process and methods of those patents, applications and other documents may be selected for the invention and embodiments thereof.

1. A method for analyzing cells in a mixture of a plurality of cells, the method comprising:
providing a plurality of examination areas wherein at least one examination area includes at least one cell to be analyzed, at least one of a plurality of multi-indexed barcodes, and at least one of a plurality of capture beads, wherein each capture bead further comprises a plurality of oligonucleotides;
performing an assay in at least one of the plurality of examination areas;
collecting assay information from the examination area;
performing release-and-capture in the at least one examination area, wherein the release-and-capture comprises:
releasing nucleic acid from the at least one cell into the examination area such that at least one of the released nucleic acids is captured by at least one of the plurality of oligonucleotides attached to at least one of the plurality of capture beads, and
releasing at least one of the indices from the at least one multi-indexed barcode in the examination area such that the released index is captured by at least one oligonucleotide attached to the at least one capture bead;
analyzing the at least one capture bead, wherein the analysis comprises;
identifying at least one nucleic acid captured from the cell, and
identifying at least one captured index from the multi-index barcode; and
generating a molecular-functional profile of the cell in the examination area,
wherein each multi-indexed barcode, of the plurality of multi-indexed barcodes, comprises at least two indices, and
wherein at least one of the plurality of oligonucleotides on the at least one capture bead attaches to at least one nucleic acid fragment released from the at least one cell in the examination area and to at least one index from at least one of the plurality of multi-indexed barcodes.

2. The method of claim 1, further comprising isolating one or more examination areas from others of the plurality of examination areas by at least one of: fluidic isolation, mechanical isolation, physical isolation, chemical isolation, thermal isolation, optical isolation, and/or genetic isolation.

3. The method of claim 2, wherein the isolating the one or more examination areas further comprises at least one of droplet isolation, bead isolation, microwell isolation, nanowell isolation, picowell isolation, isolation in aqueous media surrounded by oil media, isolation in confined physical geometries comprising aqueous media capped by mechanical, and/or providing oil or air barriers.

4. The method of claim 1, wherein at least two of the plurality of examination areas are fluidically connected.

5. The method of claim 1, wherein at least one of the plurality of examination areas is a split examination area, and wherein the split examination area is an examination area split into multiple examination areas.

6. The method of claim 5, wherein two or more of the multiple examination areas of the split examination area are fluidically connected.

7. The method of claim 1, wherein the plurality of cells comprises cells of at least one cell type of mammalian cells, eukaryotic cells, bacterial cells, fungal cells, plant cells, prokaryotic cells, immune cells, cancer cells, antibody secreting cells and/or genetically modified cells.

8. The method of claim 1, wherein the multiple indices of the multi-index barcode comprise at least two of an optical index, an oligonucleotide index, a mass index, a charge index, a size index, a fluorescence index, a chemical index, a shape index, a hardness index, an ionization index, a nucleic acid index, a smell index, and/or an audio index.

9. The method of claim 1, wherein the multiple indices of the multi-index barcodes are bound to a common substrate, wherein the substrate comprises at least one of a solid substrate, a gel substrate, a dissolvable substrate, a crosslinked substrate, a nucleotide substrate, a chemical substrate, a droplet substrate, a biological substrate and/or a physical substrate.

10. The method of claim 9, wherein the multiple indices are bound to the substrate via linkers, and one or more of the linkers are cleavable.

11-35. (canceled)

36. A method for analyzing a plurality of capture beads, the method comprising:
exposing the plurality of capture beads to a plurality of cells, wherein at least one of the plurality of cells is in at least one examination area of a plurality of examination areas;
collecting the plurality of capture beads, wherein each capture bead comprises at least one of:
a perturbation agent,
a capture oligonucleotide,
genetic content captured from the at least one cells exposed to the bead in the at least one examination area,
a multi-index barcode,
a spatial-index barcode, and/or
a plurality of non-nucleotide tags;
sequencing a capture bead of the plurality of capture beads; and
generating an identity associated with the capture bead, wherein the generated identity comprises a sequence of the genetic content captured from the at least one cell and a sequence of the capture oligonucleotide,
wherein sequencing the capture bead comprises sequencing the capture oligonucleotide and sequencing the genetic content, and
wherein the sequence of the genetic content comprises nucleic acid captured from the at least one cell exposed to the capture bead, and the generated identity further comprises synthesis history of the perturbation agent on the capture bead and/or spatial location of the at least one cell exposed to the capture beads.

37-76. (canceled)

77. A method for single-cell barcoding using non-distinguishable beads, the method comprising:
introducing at least one non-distinguishable bead to an examination area, wherein the examination area has at least one cell of interest;
liberating nucleic acid content from the cell of interest into the examination area;
capturing the nucleic acid content on the non-distinguishable bead;
extracting the non-distinguishable bead from the examination area; and
introducing at least one barcode to the non-distinguishable bead, wherein the barcode is attached to the nucleic acid content captured on the non-distinguishable bead and the barcode is unique to the non-distinguishable bead.

78-84. (canceled)

85. A method for single-cell barcoding comprising:
providing at least one examination area, the examination area comprising:
at least one cell to be analyzed, and
at least one non-distinguishable capture bead;
lysing the cell in the examination area to extract nucleic acid content from the cell;
capturing at least a portion of the nucleic acid content on the non-distinguishable capture bead;
extracting the non-distinguishable capture bead from the examination area; and
synthesizing at least one unique barcode, wherein the unique barcode is introduced to an end of the captured at least the portion of the nucleic acid content on the non-distinguishable capture bead.

86. A composition comprising a non-distinguishable solid support and a synthesized barcode, the synthesized barcode comprising:
a degenerate and/or indistinguishable capture sequence, and
captured nucleic acid content from a lysed cell,
wherein the nucleic acid content is coupled to the degenerate and/or indistinguishable capture sequence.

87-89. (canceled)

90. A method for barcoding a non-distinguishable bead, the method comprising:
exposing the non-distinguishable bead to a cell of interest;
lysing the cell of interest;
capturing, via at least one degenerate and/or indistinguishable capture sequence on the non-distinguishable bead, nucleic acid content from the cell of interest.

91. The method of claim 90, wherein the cell of interest is selected from the group consisting of: mammalian cells, eukaryotic cells, bacterial cells, fungal cells, plant cells, prokaryotic cells, immune cells, cancer cells, antibody secreting cells, and/or genetically modified cells.

92. A method for capturing positional information from single-cells using a solid support having an optical barcode, the method comprising:
providing at least one examination area, the at least one examination area including:
at least one cell to be analyzed, and
at least one capture bead, the capture bead having at least one optically distinguishable barcode, wherein the optically distinguishable barcode corresponds to the examination area;
lysing the at least one cell in the examination area, wherein the lysing of the at least one cell releases nucleic acid content;
capturing at least a portion of the nucleic acid content via the capture bead;
extracting the capture bead from the examination area;
performing ledger synthesis on the extracted capture bead, wherein the ledger synthesis produces a unique nucleotide-based barcode that corresponds to the optically distinguishable barcode.

93-95. (canceled)

96. A method for corresponding spatial information to a single cell using optically barcoded beads, the method comprising:
providing at least one examination area, wherein the examination area has at least one cell;
exposing the at least one cell to an optically barcoded bead in the examination area, wherein an optical barcode of the optically barcoded bead corresponds to the examination area;
releasing nucleic acid content from the cell,
capturing the nucleic acid content on the optically barcoded bead;
removing the optically barcoded bead from the examination area;
conducting split-pool synthesis on the optically barcoded bead, such that a unique barcode is synthesized on the optically barcoded bead, wherein the unique barcode comprises nucleotides;
coupling the unique barcode and the optical barcode of the optically barcoded bead; and
corresponding the unique barcode and the optical barcode to the examination area.

97. A method for performing spatial analysis on a single cell using ledger synthesis, the method comprising:
placing at least one cell and at least one optically barcoded bead in an examination area;
extracting nucleic acid content from the at least one cell;
capturing the nucleic acid content with the optically barcoded bead;
removing the optically barcoded bead from the examination area;
performing split-pool synthesis, wherein a unique barcode is generated and coupled to the optically barcoded bead;
tracking the optical barcode using a ledger; and
sequencing the optically barcoded bead,
wherein the unique barcode and the optical barcode correspond to the examination area.

98. A composition comprising a solid support having an optical barcode, the solid support further comprising nucleic acid content of a lysed cell and a plurality of unique nucleotide fragments, wherein the plurality of unique nucleotide fragments forms a unique barcode, and the unique barcode corresponds to the optical barcode of the solid support.

99-101. (canceled)
